(19) United States
(12) Patent Application Publication
(10) Pub. No.: US 2016/0244520 A1
(43) Pub. Date: Aug. 25, 2016

(54) COMPOSITIONS AND METHODS FOR TREATING PSORIATIC ARTHRITIS

(71) Applicant: Abbvie Inc., North Chicago, IL (US)

(72) Inventors: Robert J. Padley, Lake Bluff, IL (US); Paul M. Peloso, Gurnee, IL (US); Ahmed A. Othman, Waukegan, IL (US)

(21) Appl. No.: 15/004,791

(22) Filed: Jan. 22, 2016

Related U.S. Application Data
(60) Provisional application No. 62/107,389, filed on Jan. 24, 2015.
(60) Provisional application No. 62/152,817, filed on Apr. 24, 2015.
(60) Provisional application No. 62/219,634, filed on Sep. 16, 2015.

(51) Int. Cl. C07K 16/24; A61K 9/00; A61K 9/19; A61K 47/48; A61K 39/395; A61K 31/519

(52) U.S. Cl. CPC C07K 16/244; C07K 16/241; A61K 39/3955; A61K 31/519; A61K 9/19; A61K 47/48676; A61K 9/0019; C07K 2317/31; C07K 2317/56; C07K 2317/76; A61K 2039/505

(57) ABSTRACT

Proteins that bind IL-17 and TNF-α are described along with their use in compositions and methods for treating psoriatic arthritis.

### RELATED APPLICATIONS

The instant application claims the benefit of priority to U.S. provisional application Ser. No. 62/107,389 filed Jan. 24, 2015, U.S. provisional application Ser. No. 62/152,817 filed Apr. 24, 2015, and U.S. provisional application Ser. No. 62/219,634 filed Sep. 16, 2015, the contents of which are hereby incorporated by reference in their entireties.

### FIELD OF THE INVENTION

The present invention relates to bispecific TNF and IL-17 binding protein compositions, and to their uses in the prevention and/or treatment of psoriatic arthritis.

### BACKGROUND OF THE INVENTION

Psoriatic arthritis (PsA) is defined as a unique inflammatory arthritis affecting the joints and connective tissue, and is associated with psoriasis of the skin or nails. It is a hyperproliferative and inflammatory arthritis that is distinct from rheumatoid arthritis (RA). The etiology of PsA is not fully understood. Genetic susceptibility and exogenous influences may play roles in the cause of the disease, potentially causing pain symptoms to flare and subside, vary from person to person, and even change locations in the same person over time.

PsA can affect any joint in the body, and may affect one or multiple joints. Affected fingers and toes can become extremely swollen, a condition often referred to as dactylitis. PsA in the spine, called spondylitis, causes pain in the back or neck and difficulty bending. PsA also can cause tender spots where tendons and ligaments join to bones. This condition, called enthesitis, can result in pain at the back of the heel, the sole of the foot, around the elbows or in other areas and is one of the characteristic features of PsA.

PsA treatments vary depending on the level of pain. Those with very mild arthritis may require treatment only when their joints are painful and may stop therapy when symptoms improve. Non-steroidal anti-inflammatory drugs such as ibuprofen (Motrin® or Advil®) or naproxen (Aleve®) are often used as an initial treatment. If the arthritis does not respond, disease modifying anti-rheumatic drugs (DMARDs) may be prescribed. These include sulfasalazine (Azulfidine®), methotrexate (Rheumatrex®), cyclosporine (Neoral®, Sandimmune®) and leflunomide (Arava®). Sometimes combinations of these drugs may be used together. The anti-malarial drug hydroxychloroquine (Plaquenil®) has been shown effective, but it usually is avoided as it can cause a flare-up of psoriasis. Azathioprine (Imuran®) has been shown to be effective those with severe forms of PsA. For swollen joints, corticosteroid injections can be useful. Surgery can be helpful to repair or replace badly damaged joints.

There remains a need in the art for therapeutics that effectively and safely treat PsA including those cases characterized by resistance to treatment using DMARDs such as methotrexate as well as other binding protein treatments.

### SUMMARY OF THE INVENTION

This disclosure provides methods for treating a subject having psoriatic arthritis (referred to herein as PsA), the method comprising the step of administering to the subject a binding protein that specifically binds both human interleukin 17 (IL-17) and human tumor necrosis factor (TNF, also known as TNF-α), wherein the binding protein is a dual variable domain immunoglobulin (DVD-Ig) binding protein. In various embodiments, the subject is resistant to treatment with at least one disease-modifying antirheumatic drug (DMARD). In various embodiments, the DMARD comprises a biological molecule or agent, for example a protein having an amino acid sequence. In various embodiments, the DMARD comprises a non-biologic molecule or agent. For example, the DMARD can be a small molecule. In various embodiments, the DMARD comprises methotrexate (MTX). In various embodiments, the DMARD comprises sulfasalazine, cyclosporine, leflunomide, hydroxychloroquine, or azathioprine. In various embodiments, the method further includes administering to the subject a DMARD. For example, the DMARD can be administered prior to or concurrently with the binding protein. Alternatively, the DMARD is administered subsequent to the binding protein being administered. In various embodiments, the subject is receiving a dose of the DMARD, comprising an amount of less than 10 mg per week. Optionally, the subject is a male or a female who has been diagnosed with PsA for a period of time, e.g., days or weeks. In various embodiments, the subject has been diagnosed with PsA for at least three months. In various embodiments, the subject has been on a stable regimen of the DMARD for a period of time, e.g., days, weeks or months. In certain embodiments, the subject has been on a DMARD for at least four weeks. In various embodiments, the stable DMARD regimen comprises a dose less than or equal to 10 mg per week.

In various embodiments, the binding protein neutralizes TNF-α and/or IL-17 in vivo. In various embodiments, the PsA affects one joint, two joints, three joints, four joints, or five joints. In various embodiments, the PsA is manifested in the subject by one or more symptoms selected from stiffness, pain, swelling, tenderness of the joints, and tenderness of the area surrounding a ligament or a tendon. In various embodiments, the PsA is experienced and/or diagnosed in a knee, hip, hand, finger, spine/back, toe, and/or foot. In various embodiments, the subject suffering from PsA has pain, e.g., tendon pain. In various embodiments, the subject has at least one joint or nail deformity. In various embodiments, the methods of the invention result in treatment of or amelioration of at least one of the symptoms of PsA. In various embodiments, symptoms of PsA comprise enthesitis or dactylitis.

In various embodiments, the binding protein comprises a heavy chain variable region (VH) for binding TNF-α comprising the amino acid sequence of SEQ ID NO: 5. In various embodiments, the binding protein comprises a VH for binding IL-17 comprising the amino acid sequence of SEQ ID NO: 7.

In various embodiments, the binding protein comprises a light chain variable region (VL) for binding TNF-α comprising the amino acid sequence of SEQ ID NO: 10. In various embodiments, the binding protein comprises a VL for binding IL-17 comprising the amino acid sequence of SEQ ID NO: 12.

In a related embodiment, the binding protein comprises a heavy chain that binds both TNF-α and IL-17 comprising the amino acid sequence of SEQ ID NO: 4 and a light chain that binds both TNF-α and IL-17 comprising the amino acid sequence of SEQ ID NO: 9. In various embodiments, the binding protein comprises a peptide linker. In certain embodiments, the peptide linker comprises the amino acid sequence of SEQ ID NO: 6, SEQ ID NO: 11, or a portion or a combination thereof. In various embodiments the peptide linker comprises the amino acid sequence of at least one of SEQ ID NOs: 14-48.

In certain embodiments, the binding protein comprises a heavy chain comprising an amino acid sequence that is greater than 80%, 85%, 90%, 95%, 96%, 98%, 99%, or 99% identical to the amino acid sequence of SEQ ID NO: 4, and/or a light chain comprising an amino acid sequence that is greater than 80%, 85%, 90%, 95%, 96%, 98%, 99%, or 99% identical to the amino acid sequence of SEQ ID NO: 9. In a related embodiment, the binding protein comprises 3 CDRs identical to the amino acid sequence of the corresponding 3 CDRs in the amino acid sequence of SEQ ID NO: 4. In a related embodiment, the binding protein comprises 3 CDRs identical to the amino acid sequence of the corresponding 3 CDR in the amino acid sequence of SEQ ID NO: 9.

In various embodiments, the binding protein comprises a constant region described herein, for example, in Table 3. In certain embodiments, the binding protein comprises at least one heavy chain constant region, at least one light chain constant region or at least one heavy chain and one light chain constant region. In one embodiment, the heavy chain constant region comprises the amino acid sequence of SEQ ID NO: 8. In another embodiment, the light chain constant region comprises the amino acid sequence of SEQ ID NO: 13. In various embodiments, the constant region comprises at least one amino acid mutation. In certain embodiments, the mutation comprises at least one amino acid change, deletion or insertion in the amino acid sequences of SEQ ID NOs: 8 or 13.

In various embodiments, the binding protein is formulated in a pharmaceutical composition comprising a pharmaceutically acceptable carrier. In various embodiments, the binding protein is crystallized. In various embodiments, the crystallized binding protein is formulated in a composition comprising at least one excipient and/or a polymeric carrier. For example, the at least one polymeric carrier is a polymer selected from the group consisting of poly (acrylic acid), poly (cyanoacrylate), poly (amino acid), poly (anhydride), poly (depsipeptide), poly (ester), poly (lactic acid), poly (lactic-co-glycolic acid) or PLGA, poly (b-hydroxybutryate), poly (caprolactone), poly (dioxanone), poly (ethylene glycol), poly (hydroxypropyl) methacrylamide, poly [(organo)phosphazenel, poly (ortho ester), poly (vinyl alcohol), poly (vinylpyrrolidone), maleic anhydride-alkyl vinyl ether copolymer, pluronic polyol, albumin, alginate, cellulose, cellulose derivative, collagen, fibrin, gelatin, hyaluronic acid, oligosaccharide, glycaminoglycan, sulfated polysaccharide, blend, and copolymer thereof. In various embodiments, the subject is also administered a pain reliever, or a nonsteroidal anti-inflammatory drug (NSAID). Alternatively, the subject is administered a steroid. In various embodiments, the at least one excipient is selected from the group consisting of albumin, sucrose, trehalose, lactitol, gelatin, hydroxypropyl-β-cyclodextrin, methoxypolyethylene glycol, and polyethylene glycol.

In various embodiments, the binding protein is formulated in a composition comprising at least one of a buffer, a polyol and a surfactant. For example, the binding protein is formulated in a composition comprising an acetate buffer, citrate buffer, phosphate buffer, or a histidine buffer. In various embodiments, the binding protein is formulated in a composition comprising sucrose or sorbitol. In various embodiments, the surfactant includes sodium lauryl sulfate, a polysorbate such as polysorbate 20, polysorbate 40, polysorbate 60, and polysorbate 80. In various embodiments, the binding protein is formulated as a powder or lyophilisate. In certain embodiments, water is added to the powder to form a reconstituted solution. In various embodiments, the reconstituted solution comprising the binding protein is administered as an injection. In various embodiments, acid added as necessary to adjust pH. In various embodiments, the binding protein is reconstituted with 0.5-5 milliliters (ml or mL) of sterile water for the injection. In various embodiments, the binding protein being reconstituted is at a concentration of between 10 and 200 mg/ml.

In various embodiments, the binding protein is administered subcutaneously. In various embodiments, the binding protein is administered intravenously. In various embodiments, the binding protein is administered at a dosage/dose of about 0.1 milligram per kilogram of subject weight (mg/kg), 0.3 mg/kg, 1.0 mg/kg, 2 mg/kg, 3 mg/kg, 4 mg/kg, 5 mg/kg, 6 mg/kg, 7 mg/kg, 8 mg/kg, 9 mg/kg, or 10 mg/kg. For example, the dose is administered at a dose from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 1.5 mg/kg, from 1.5 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. In various embodiments, the binding protein is subcutaneously administered at a dose of about 1.5 mg/kg. In various embodiments, the binding protein is subcutaneously administered at a dose of about 0.3 mg/kg, 1 mg/kg, 3 mg/kg, or 10 mg/kg.

In various embodiments, the binding protein is administered at a total dose of between 1-25 mg, 25-50 mg, 50-75 mg, 75-100 mg, 100-200 mg, 100-125 mg, 125-150 mg, 150-175 mg, 175-200 mg, 200-225 mg, 225-250 mg, 250-275 mg, 275-300 mg, 300-325 mg, or 325-350 mg of the binding protein. In a certain embodiment, the binding protein is subcutaneously administered weekly at a dose of about 120 mg. In a certain embodiment, the binding protein is subcutaneously administered weekly at a dose of about 240 mg.

The binding protein may be administered using different regimens and administration schedules. For example, the binding protein may be administered once or a plurality of times (e.g., twice, three times, four times to eight times, eight times to ten times, and ten times to twelve times). For example the administration schedule is determined based on the efficacy and/or tolerability of the binding protein in the individual or subject. In various embodiments, the binding protein is administered at least once, for example every day, every other day, every week, every two weeks, every three weeks, every four weeks, and every month. In certain embodiments, the binding protein is administered every week at a dose of about 0.3 mg/kg, 1.0 mg/kg, 1.5 mg/kg, 3 mg/kg, or 10 mg/kg. In various embodiments, the binding protein is administered at a weekly total dose of about 25-375 mg. In certain embodiments, the binding protein is administered at a dose of about 200-280 mg per week. In some embodiments, the binding protein is subcutaneously administered weekly at a dose of about 240 mg.

In various embodiments, the subject has been treated with a DMARD for a period of time prior to administration of the binding protein and the subject has become resistant to the DMARD treatment/therapy. For example, the DMARD resistance comprises the worsening of at least one symptom associated with PsA wherein the DMARD dose is maintained at the same level or increased. In various embodiments, the binding protein modulates and reduces the level of resistance by improving at least one symptom associated with DMARD resistance wherein the DMARD dose is maintained or decreased.

In various embodiments, the method further includes administering the binding protein after administering the DMARD. In one embodiment, the DMARD is methotrexate. Alternatively, the method involves administering the binding protein prior to or concurrently with the DMARD. In a related embodiment of the method, administering the binding protein improves at least one negative condition in the subject associated with PsA or PsA-associated symptom in the subject. In various embodiments, the at least one PsA-associated symptom is selected from the group consisting of an autoimmune response (e.g., antibodies and adverse effects), inflammation, stiffness, pain, bone erosion, osteoporosis, joint deformity, joint destruction, a nerve condition (e.g., manifested in tingling, numbness, and burning), scarring, a cardiac disorder/condition, a blood vessel disorder/condition, high blood pressure, fatigue, anemia, weight loss, abnormal body temperature, fever, a lung condition/disease, a kidney condition/disorder, a liver condition/disorder, an ocular disorder/condition, a skin disorder/condition, an intestinal disorder/condition, and an infection.

In various embodiments, administration of the binding protein to the subject improves a score of one or more PsA metrics or criteria in the subject. In various embodiments, the PsA metric is selected from the group consisting of American College of Rheumatology Response Rate (ACR for example ACR20, ACR50, and ACR70); proportion of subjects achieving Low Disease Activity (LDA); Disease Activity Score 28 (DAS28; e.g., DAS28 based on C-reactive protein); swollen joints; tender joints patient assessments of pain; global disease activity and physical function; physician global assessment of disease activity and acute phase reactant; Psoriasis Area and Severity Index (PASI); plaque erythema; plaque scaling and plaque thickness; assessment of dactylitis; Entheses Sites Comprising the Total Spondyloarthritis Research Consortium of Canada (SPARCC); Enthesitis Index; Self-Assessment of Psoriasis Symptoms (SAPS); quality of life, function and work as measured by the SF36v2; quality of life by self-reporting by the patient/subject; change in the quality of life, function and work as measured by Bath AS Disease Activity Index (BASDAI); quality of life, function and work as measured by the Fatigue Numeric Rating Scale; quality of life, function and work as measured by the Sleep Quality Scale; psoriatic arthritis disease activity score (PASDAS); psoriasis target lesion score; proportion of subjects achieving ACR70 responder status; and classification of psoriatic arthritis (CASPAR). In certain embodiments, the binding protein improves the PsA metric or criteria by at least 1%, 3%, 5%, 7%, 10%, 15%, 20%, 25%, 30%, 35%, 40%, 45%, 50%, 55%, 60%, 65%, 70%, 75%, 80%, 85%, 90%, 95%, or 99%.

In various embodiments, the method further comprises observing or detecting a modulation (e.g., reduction or increase) in the expression or activity of a biomarker. In various embodiments, the biomarker comprises a skin biomarker or a biopsy biomarker. In various embodiments, the biomarker is selected from the group consisting of high-sensitivity C-reactive protein (hsCRP), a matrix metallopeptidase (MMP, e.g., MMP-9), vascular endothelial growth factor (VEGF), a MMP degradation product (e.g., an MMP degradation product of type I, II, or III collagen (C1M, C2M, C3M)), C-reactive protein (CRP), a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), substance P, an inducible Nitric Oxide Synthase (iNOS), cartoxin I (CTX-I), cartoxin II (CTX-II), type II collagen neoepitope (TIINE), creatinine, and a vimentin (e.g., a citrullinated and MMP-degraded vimentin; VICM). In various embodiments, the binding protein reduces the arthritis and/or modulates (e.g., reduces and increases) expression and/or activity of the biomarker by at least 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

This disclosure also provides methods for treating a subject having PsA, wherein the subject is resistant to treatment with methotrexate, the method comprising the step of administering to the subject a composition comprising a binding protein that specifically binds both IL-17 and TNF-α, wherein the binding protein is a DVD-Ig protein, and wherein the binding protein comprises at least one polypeptide comprising an amino acid sequence of SEQ ID NO:4 and an amino acid sequence of SEQ ID NO:9, wherein the binding protein is administered weekly and the total amount administered is about 120 mg or about 240 mg of the binding protein. The binding protein in various embodiments comprises a constant region described herein for example in Table 3. In various embodiments, the constant region comprises at least one mutation compared to a wild-type constant region. In various embodiments, the heavy chain constant region comprises the amino acid sequence of SEQ ID NO: 8. In various embodiments, the light chain constant region comprises the amino acid sequence of SEQ ID NO: 13. In various embodiments, the binding protein is subcutaneously administered weekly at a dose of about 120 mg. In a related embodiment, the binding protein is subcutaneously administered weekly at a dose of about 240 mg.

This disclosure also provides methods of treating a subject having PsA, wherein the subject has been, or is currently being treated with methotrexate, the method comprising administering to the subject a binding protein that binds both TNF-α and IL-17, wherein the binding protein is a DVD-Ig binding protein, wherein the binding protein comprises a heavy chain comprising the amino acid sequence of SEQ ID NO: 4 and comprises a light chain comprising the amino acid sequence of SEQ ID NO: 9, wherein the binding protein is administered at a dose from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 1.5 mg/kg, from 1.5 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. In various embodiments, the binding protein is administered at a dose of about 1.5 mg/kg. In various embodiments of the method, the binding protein is administered at a dose of about 3.0 mg/kg. In various embodiments, the binding protein is administered intravenously or subcutaneously. In various embodiments, the binding protein is administered at least once per day. In various embodiments of the method, the binding protein is administered every day, every other day, every week, every two weeks, every three weeks, every four weeks, or every month.

This disclosure also provides methods for treating a subject having PsA wherein the subject has or is currently being treated with methotrexate, the method comprising: administering to the individual a binding protein that binds both TNF-α and IL-17, wherein the binding protein is a DVD-Ig binding protein, wherein the binding protein comprises a heavy chain comprising the amino acid sequence of SEQ ID NO: 4, and comprises a light chain comprising the amino acid sequence of SEQ ID NO: 9, wherein administering the binding protein is performed using multiple individual doses to reach the total dose. In various embodiments, the total dose is a weekly total dose and is between 1-25 mg, 25-50 mg, 50-75 mg, 75-100 mg, 100-200 mg, 100-125 mg, 125-150 mg, 150-175 mg, 175-200 mg, 200-225 mg, 225-250 mg, 250-275 mg, 275-300 mg, 300-325 mg, or 325-350 mg of the binding protein. In certain embodiments, the weekly total dose is about 120 mg or 240 mg. In various embodiments, the binding protein is administered at least once per day. In various embodiments of the method, the binding protein is administered every day, every other day, every week, every two weeks, every three weeks, every four weeks, or every month.

In various embodiments, the binding protein comprises a constant region. For example, the constant region is one described herein, for example in Table 3. In certain embodiments, the binding protein comprises at least one heavy chain constant region, at least one light chain constant region or at least one heavy chain and one light chain constant region. In one embodiment, the heavy chain constant region comprises the amino acid sequence of SEQ ID NO: 8. In another embodiment, the light chain constant region comprises the amino acid sequence of SEQ ID NO: 13.

In various embodiments, the binding protein is administered intravenously or subcutaneously. In various embodiments, the binding protein is administered after administering methotrexate. Alternatively, the binding protein is administered concurrently or prior to administering methotrexate.

In various embodiments, the binding protein is administered at a dosage/dose of about 0.1 mg/kg, 0.3 mg/kg, 1.0 mg/kg, 1.5 mg/kg, 3 mg/kg and 10 mg/kg. In various embodiments, the subcutaneous dose of binding protein is approximately 1.5 mg/kg. In various embodiments, the subcutaneous dose of binding protein is approximately 3.0 mg/kg.

In various embodiments, the method of treating further comprises identifying an improvement in the subject in regards to the severity or duration of a symptom associated with the PsA. In certain embodiments, identifying an improvement in the subject in regards to the severity or duration of a symptom associated with the PsA comprises using one or more scores, tests, or metrics for PsA or inflammation. In various embodiments, the score, test, or metric is selected from the group consisting of an ACR score, for example, ACR20, ACR50, and ACR70; proportion of subjects achieving LDA; DAS28; swollen joints; tender joints; patient assessments of pain; global disease activity and physical function; physician global assessment of disease activity and acute phase reactant levels; PASI, plaque erythema; plaque scaling; and plaque thickness; assessment of dactylitis; SPARCC; Enthesitis Index; SAPS; quality of life, function and work as measured by the SF36v2; quality of life by self-reporting; change in the quality of life, function and work as measured by BASDAI; quality of life, function and work as measured by the Fatigue Numeric Rating Scale; quality of life, function and work as measured by the Sleep Quality Scale; PASDAS; Psoriasis Target Lesion Score; Proportion of subjects achieving ACR70 responder status; and CASPAR.

The binding protein in various embodiments comprises a constant region described herein. In various embodiments, the heavy chain constant region comprises the amino acid sequence of SEQ ID NO: 8. In various embodiments, the light chain constant region comprises the amino acid sequence of SEQ ID NO: 13.

In certain embodiments, a composition comprising the binding protein comprises a lyophilized material, or a re-constituted material from a lyophilized material; and/or wherein the composition is sterile. In some embodiments, the composition comprises a fluid or a suspension. In some embodiments, the binding protein comprises a crystallized protein or a conjugate.

In certain embodiments, the binding protein is administered at least twice. In some embodiments, prior to administering the binding protein the subject was diagnosed to have a resistance to a DMARD.

In some embodiments, administering the binding protein reduces a negative condition and/or modulates a biomarker associated with the PsA. In some embodiments, the binding protein neutralizes TNF-α and IL-17 for a period of time. Optionally, the period of time is selected from the group consisting of about 4 hours, 12 hours, 1 day, 2 days, 3 days, 4 days, 10 days, 15 days, 18 days, 21 days, 36 days, 48 days, 60 days, 72 days, and 84 days.

In certain embodiments, the methods described herein further comprise the step of detecting modulation of a TNF-α mediated symptom or an IL-17-mediated symptom.

The disclosure also provides a dose of a bispecific binding protein that neutralizes TNF-α and IL-17 sufficient to treat or prevent at least one symptom of PsA. In certain embodiments, the dose comprises about 120 mg or about 240 mg of bispecific binding protein. In certain embodiments, the binding protein comprises a VH for binding TNF-α comprising the amino acid sequence of SEQ ID NO: 5 and a VH for binding IL-17 comprising the amino acid sequence of SEQ ID NO: 7. In certain embodiments, the binding protein comprises the amino acid sequence of SEQ ID NO: 4.

In other embodiments, the binding protein comprises a VL for binding TNF-α comprising the amino acid sequence of SEQ ID NO: 10 and a VL for binding IL-17 comprising the amino acid sequence of SEQ ID NO: 12. In certain embodiments, the binding protein comprises the amino acid sequence of SEQ ID NO: 9. In certain embodiments the binding protein comprises a heavy chain comprising the amino acid sequence of SEQ ID NO: 4 and a light chain comprising the amino acid sequence of SEQ ID NO: 9. In some embodiments, the binding protein further comprises a constant region.

In certain embodiments, the binding protein comprises a conjugate with a second agent. Optionally, the second agent is selected from the group consisting of an immunoadhesion molecule, an imaging agent, a therapeutic agent, and a cytotoxic agent.

### BRIEF DESCRIPTION OF THE DRAWINGS

FIG. 1 is a graph showing total number of subjects (ordinate) as a function of the visit day for study M12-962 (abscissa). Subjects were subcutaneously administered 1.5 mg/kg of ABT-122 per week (8 total doses during study). The highlighted areas show the percentage of subjects at each visit having achieved the desired positive result of having clear/almost clear skin on Physician Global Assessment of Disease Activity or 75% reduction in Psoriasis Area and Severity Index (PASI) score from baseline).

FIG. 2 is a graph showing total number of subjects (ordinate) as a function of the visit day for study M12-962 (abscissa). Subjects were subcutaneously administered 1.5 mg/kg of ABT-122 per week (8 total doses during study). The highlighted areas show the percentage of subjects at each visit having achieved the desired positive result of 75% reduction in Psoriasis Area and Severity Index (PASI) score from baseline.

FIG. 3 is a graph showing Physician Global Assessment of Disease Activity scores for individual subjects on each visit (ordinate) as a function of the visit day for study M12-962 (abscissa). The blinded subjects were assigned/identified as numbers 3962, 4160, 4259, 4655, 4853, 4952, 5051 and 5249.

FIG. 4 is a graph showing PASI scores for individual subjects on each visit (ordinate) as a function of the visit day for study M12-962 (abscissa). The blinded subjects were assigned/identified as numbers 3962, 4160, 4259, 4655, 4853, 4952, 5051 and 5249.

FIG. 5 is a schematic of a Phase 2, randomized, double-blind, double-dummy, active- and placebo-controlled, parallel-group multicenter study described herein. This study is designed to assess the safety, tolerability, efficacy, pharmacokinetics and immunogenicity of varying doses of ABT-122 administered on background methotrexate (MTX). The screening period lasted up to 30 days, and on day 1 patients are randomized. The 12 week double-blind treatment period begins with patients being subcutaneously injected with either 240 mg of ABT-122 every week (EW; n=66); 120 mg of ABT-122 every week (n=66); 40 mg of Humira® antibody every other week (n=66); or a placebo every week (i.e., no ABT-122; n=22).

FIG. 6 is a schedule of the system and regimen used to analyze adverse effects for patients receiving ABT-122 in the study described in FIG. 5.

### DETAILED DESCRIPTION OF THE INVENTION

ABT-122 is an IgG1 dual-variable domain immunoglobulin (DVD-Ig) binding protein described herein that specifically binds and neutralizes the pro-inflammatory cytokines TNF-α and IL-17 and prevents them from binding to their respective receptors on cells. TNF is typically a soluble homotrimer, after being enzymatically cleaved from the cell surface (Tracey et al. (2008) Pharmacol Ther. 117 (2):244-79). ABT-122, binds to IL-17A, thereby neutralizing IL-17A homodimers and IL-17A-F heterodimers but not to other members of the IL-17 family. ABT-122 has two sets of variable domain sequences connected in tandem by flexible peptide linkers, and includes human immunoglobulin G1 (IgG1) heavy chain and κ light chain constant regions. Human IgG1 molecules found in nature are bivalent and monospecific with a molecular weight of approximately 150 kilodaltons. In ABT-122, the heavy and light chains form a tetravalent, bi-specific immunoglobulin-like molecule with a molecular weight of 198 kilodaltons.

TNF and IL-17 have important roles in the pathogenesis of PsA and other inflammatory diseases. Both cytokines are expressed at increased levels in synovial tissue and are key factors in the joint inflammation and damage to bone and cartilage that are hallmarks of the disease (Frleta et al. (2014) Curr. Rheumatol. Rep. 16(4):414). TNF blockade is an established therapy for PsA. IL-17 blockade has demonstrated efficacy in psoriasis (Langley et al. (2014) N. Engl. J. Med. 371(4):326-38; Papp et al. (2012) N. Engl. J. Med. 366(13):1181-9; Tham et al. (2014) J. Clin. Pharmacol. 54(10):1117-24). Clinical trials are currently being conducted for PsA and other inflammatory diseases (Gisondi et al. (2014) Dermatol. Ther. (Heidelb) 4(1):1-9; McInnes et al. (2014) Ann. Rheum. Dis. 73(2):349-56; Mease et al. (2014) N. Engl. J. Med. 370(24):2295-306). Without being limited by any particular theory or mechanism of action, it is here envisioned that ABT-122 binding protein as described herein is as effective as or more effective than current treatments for PsA.

In the first-in-human, single ascending dose study of ABT-122 (Study M12-704), 48 healthy volunteers were administered a single dose of ABT-122, ranging from 0.1 mg/kg to 10 mg/kg by intravenous (IV) administration and 0.3 mg/kg to 3 mg/kg by subcutaneous (SC) administration. See international patent publication WO2015/138337, which is incorporated by reference herein in its entirety. No events of severe intensity, serious adverse events, systemic hypersensitivity reactions or injection site reactions, discontinuations due to adverse events, or deaths occurred. There were no dose limiting toxicities and no apparent association of particular adverse events with dose or route of administration. Following SC administration, the absolute bioavailability of ABT-122 was ˜50% and the maximum serum concentrations were observed three to four days after dosing. The majority of subjects had detectable anti-drug antibodies (ADA) across all dose groups, although they largely exhibited low titer values. The presence of ADA did not appear to influence drug clearance for the majority of the subjects and did not correlate with any systemic or serious adverse event profiles.

Examples herein show a Phase 2 study that evaluates safety, tolerability and efficacy of ABT-122 in subjects diagnosed with PsA who have signs and symptoms of active disease and are on stable methotrexate (MTX) therapy.

The present invention provides methods for treating PsA in a subject. Generally, the subject is a mammalian subject for example a human subject. In various embodiments the subject has PsA and is resistant to treatment with one or more DMARDs. Such methods comprise administering to a subject one or more binding proteins that bind both IL-17 and TNF. In another embodiment, the invention provides methods for treating PsA in a human subject using a binding protein that binds and/or neutralizes both IL-17 and TNF-α. In certain embodiments, the binding protein is a DVD-Ig binding protein. In other embodiments, administering the binding protein improves a score of one or more PsA metrics or criteria. In various embodiments, the DMARD is methotrexate. In various embodiments, the binding protein neutralizes TNF and/or IL-17 in vivo. In various embodiments, the binding protein modulates one or more negative effects of TNF and/or IL-17 in vivo for a period of time after administration of a dose. For example, the period of time can be at least four hours, twelve hours, one day, three days, a week, two weeks, three weeks, or a month.

In various embodiments, the binding protein comprises the CDR amino acid sequences of the amino acid sequence of SEQ ID NO: 4, or comprises the amino acid sequence of SEQ ID NO: 4. In other embodiments, the binding protein comprises the CDR amino acid sequences of the amino acid sequence of SEQ ID NO: 9, or comprises the amino acid sequence of SEQ ID NO:9. In one embodiment, the binding protein comprises the CDR amino acid sequences of the amino acid sequence of SEQ ID NO: 4, or comprises the amino acid sequence of SEQ ID NO: 4 and comprises the CDR amino acid sequences of the amino acid sequence of SEQ ID NO: 9, or comprises the amino acid sequence of SEQ ID NO: 9. In various embodiments, the binding protein is administered every day, every few days, every week, every other week, or every month.

In a related embodiment, the binding protein comprises the amino acid sequence of SEQ ID NO: 4 and the amino acid sequence of SEQ ID NO: 9. In a related embodiment, the binding protein comprises a heavy chain constant region comprising the amino acid sequence of SEQ ID NO: 8. In a related embodiment of the method, the binding protein comprises a light chain constant region comprising the amino acid sequence of SEQ ID NO:13.

In various embodiments, the binding protein is administered at about 60 mg every other week, about 120 mg per week, or about 120 mg every other week. In various embodiments, the binding protein is administered every week for example between 50-100 mg, 100-150 mg, 150-200 mg, 200-250 mg, 250-300 mg, or 300-350 mg. In certain embodiments, the binding protein is administered at a dose of about 240 mg per week. In various embodiments, the binding protein is administered at a dose related to the mass of the patient/subject. For example the dose is calculated in milligrams of binding protein per kilogram of patient weight, or mg/kg. In various embodiments, the binding protein is administered at a dose of about 0.1 mg/kg, 0.3 mg/kg, 1.0 mg/kg, 3.0 mg/kg, or 10 mg/kg. In various embodiments, the binding protein is formulated for administration to the patient. For example, the binding protein can be lyophilized for stability, and then reconstituted with a fluid.

The methods of the invention may include the use of a “therapeutically effective amount” of the TNFα/IL-17 DVD-Ig binding protein. A “therapeutically effective amount” means an amount effective, at dosages and for periods of time necessary, to achieve the desired therapeutic result (e.g., effective treatment of psoriatic arthritis). A therapeutically effective amount of the TNFα/IL-17 DVD-Ig binding protein may be determined by a person skilled in the art and may vary according to factors such as the disease state, age, sex, and weight of the individual, pharmacokinetics, pharmacogenetics, bioavailability, and the ability of the TNFα/IL-17 DVD-Ig binding protein to elicit a desired response in the individual. A therapeutically effective amount is also determined in part by determining whether any toxic or detrimental effects of the TNFα/IL-17 DVD-Ig binding protein are outweighed by the therapeutically beneficial effects of administering the binding protein.

Administering the binding protein is performed in various embodiments using a dose of from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, from 9 mg/kg to 10 mg/kg, from 10 mg/kg to 11 mg/kg, from 11 mg/kg to 12 mg/kg, from 12 mg/kg to 13 mg/kg, from 13 mg/kg to 14 mg/kg, from 14 mg/kg to 15 mg/kg, from 15 mg/kg to 16 mg/kg, from 16 mg/kg to 17 mg/kg, from 17 mg/kg to 18 mg/kg, from 18 mg/kg to 19 mg/kg, from 19 mg/kg to 20 mg/kg, from 20 mg/kg to 21 mg/kg, from 21 mg/kg to 22 mg/kg, from 22 mg/kg to 23 mg/kg, from 23 mg/kg to 24 mg/kg, from 24 mg/kg to 25 mg/kg, from 25 mg/kg to 26 mg/kg, from 26 mg/kg to 27 mg/kg, from 27 mg/kg to 28 mg/kg, from 28 mg/kg to 29 mg/kg, from 29 mg/kg to 30 mg/kg, or from 30 mg/kg to 40 mg/kg of weight of the binding protein to weight of the individual. In various embodiments, the binding protein is administered at about 0.1 mg/kg, 0.3 mg/kg, 1 mg/kg, 3 mg/kg, or 10 mg/kg.

In certain embodiments, the binding protein is administered as a total dose at a particular point in time of between 1-25 mg, 25-50 mg, 50-75 mg, 75-100 mg, 100-200 mg, 100-125 mg, 125-150 mg, 150-175 mg, 175-200 mg, 200-225 mg, 225-250 mg, 250-275 mg, 275-300 mg, 300-325 mg, 325-350 mg, 350 mg-400 mg of the binding protein. In certain embodiments, a total dose of between 25 mg and 400 mg is administered.

Dosage regimens may be adjusted to provide the optimum desired response (i.e., a therapeutic response). In various embodiments, the subject is screened prior to the dosage being prescribed and/or administered. For example, a single dose (e.g., bolus) may be administered once, several divided doses may be administered over time, or the dose may be proportionally reduced or increased as indicated by the exigencies of the therapeutic situation. In certain embodiments, an initial dose is administered, followed by the administration of one or more subsequent doses at a later date in time. For example, an initial dose may be administered to a subject on day 1, followed by one or more subsequent doses, e.g., every week, twice a week, every two weeks, every three weeks, every four weeks, etc., for a given period of time.

The parenteral compositions may be formulated in dosage unit form for ease of administration and uniformity of dosage. Dosage unit form refers to physically discrete units suited as unitary dosages for the subject to be treated, each unit containing a predetermined quantity of active compound calculated to produce the desired therapeutic effect in association with the required pharmaceutical carrier. Dosage unit forms are dictated by the unique characteristics of the active compound and the particular therapeutic or prophylactic effect to be achieved, and the limitations inherent in the art of compounding such an active compound for the treatment an individual.

Dosage values may vary with the type and severity of the condition to be alleviated. Specific dosage regimens should be adjusted over time according to individual need. Dosage ranges set forth herein are exemplary only and are not intended to limit the scope or practice of the claimed composition.

The TNF-α/IL-17 DVD-Ig binding protein can be incorporated into pharmaceutical compositions suitable for administration to a subject. In various embodiments, the pharmaceutical composition comprises a TNF-α/IL-17 DVD-Ig binding protein and a pharmaceutically acceptable carrier. As used herein, the term “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of substances such as wetting or emulsifying agents, preservatives or buffers, which enhance the shelf life, stability, or effectiveness of the pharmaceutical composition.

Various delivery systems are known and can be used to administer the TNF-α/IL-17 DVD-Ig binding protein for preventing or treating PsA or one or more symptoms thereof. These delivery systems include encapsulation in liposomes, microparticles, and microcapsules, recombinant cells capable of expressing the antibody or antibody fragment, receptor-mediated endocytosis (see, e.g., Wu and Wu (1987) J. Biol. Chem. 262: 4429-4432), and construction of a nucleic acid as part of a retroviral or other vector.

The methods of the invention may encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The TNF-α/IL-17 DVD-Ig binding protein may be supplied either separately or mixed together in unit dosage form, for example, as a dry lyophilized powder or water free concentrate in a hermetically sealed container such as an ampoule or sachet indicating the quantity of active agent. In various embodiments where the mode of administration is infusion, the TNF-α/IL-17 DVD-Ig binding protein can be dispensed with an infusion bottle containing sterile pharmaceutical grade water or saline. In various embodiments where the mode of administration is by injection, an ampoule of sterile water for injection or saline can be provided so that the ingredients may be mixed prior to administration.

In particular, the methods of the invention also provide that one or more of the TNF-α/IL-17 DVD-Ig binding protein is packaged in a hermetically sealed container such as an ampoule or sachette indicating the quantity of the agent. In one embodiment, one or more of the TNF-α/IL-17 DVD-Ig binding protein is supplied as a dry sterilized lyophilized powder or water free concentrate in a hermetically sealed container and can be reconstituted (e.g., with water or saline) to the appropriate concentration for administration to a subject.

The compositions in various embodiments may be in a variety of forms, including for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

Therapeutic compositions typically are sterile and stable under the conditions of manufacture and storage. The composition can be formulated as a solution, microemulsion, dispersion, liposome, or other ordered structure suitable to high drug concentration. Sterile injectable solutions can be prepared by incorporating the TNF-α/IL-17 DVD-Ig binding protein in the required amount in an appropriate solvent with one or a combination of ingredients enumerated above, as required, followed by filtered sterilization. Generally, dispersions are prepared by incorporating the TNF-α/IL-17 DVD-Ig binding protein into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the TNF-α/IL-17 DVD-Ig binding protein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

In certain embodiments, the TNF-α/IL-17 DVD-Ig binding protein may be prepared with a carrier that will protect the TNF-α/IL-17 DVD-Ig binding protein against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Methods of administering the TNF-α/IL-17 DVD-Ig binding protein include, but are not limited to, parenteral administration (e.g., intradermal, intramuscular, intraperitoneal, intravenous and subcutaneous administration), epidural administration, intratumoral administration, transdermal administration (e.g., topical administration), and rectal and transmucosal administration (e.g., intranasal and oral routes)). In addition, pulmonary administration can be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244, WO 97/32572, WO 97/44013, WO 98/31346, and WO 99/66903, each of which is incorporated herein by reference their entireties. In one embodiment, the TNF-α/IL-17 DVD-Ig binding protein is administered using Alkermes AIR® pulmonary drug delivery technology (Alkermes, Inc., Cambridge, Mass., US). The TNF-α/IL-17 DVD-Ig binding protein may be administered by any convenient route, for example by infusion or bolus injection, by absorption through epithelial or mucocutaneous linings (e.g., oral mucosa, rectal, and intestinal mucosa) and may be administered together with another biologically active agent(s). Administration can be systemic or local. Local administration may be by local infusion, injection, or by means of an implant of a porous or non-porous material, including membranes and matrices, such as sialastic membranes, polymers, fibrous matrices (e.g., Tissuel®), or collagen matrices. In various embodiments, an effective amount of the TNF-α/IL-17 DVD-Ig binding protein is administered locally to the affected area of a subject to prevent or treat PsA or a symptom thereof. In various embodiments, an effective amount of the TNF-α/IL-17 DVD-Ig binding protein is administered locally to the affected area in combination with an effective amount of one or more therapies (e.g., one or more prophylactic or therapeutic agents) other than the TNF-α/IL-17 DVD-Ig binding protein to prevent or treat PsA or one or more symptoms thereof.

In other embodiments, TNF-α/IL-17 DVD-Ig binding protein may be administered by at least one mode of administration selected from intraarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intrapleural, intraprostatic, intrapulmonary, intrarectal, intrarenal, intraretinal, intraspinal, intrasynovial, intrathoracic, intrauterine, intravesical, bolus, vaginal, rectal, buccal and sublingual. For example, the binding protein is subcutaneously administered as described in any of the working examples herein. If the TNF-α/IL-17 DVD-Ig binding protein compositions are administered topically, the compositions can be formulated in the form of an ointment, membrane, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as FREON®) or in a squeeze bottle. Moisturizers or humectants can also be added to pharmaceutical compositions and dosage forms if desired. Examples of such additional ingredients are well known in the art.

The methods of the invention may comprise pulmonary administration, e.g., by use of an inhaler or nebulizer, of a composition formulated with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244, WO 97/32572, WO 97/44013, WO 98/31346, and WO 99/66903, each of which is incorporated herein by reference their entireties.

If the methods of the invention comprise intranasal, the TNFα/IL-17 DVD-Ig binding protein composition can be formulated in the form of an aerosol, spray, mist or drops. The TNFα/IL-17 DVD-Ig binding protein can be delivered in the form of an aerosol spray from pressurized packs or a nebulizer, with the use of a suitable propellant (e.g., dichlorodifluoromethane, trichlorofluoromethane, dichlorotetrafluoroethane, carbon dioxide). In the case of a pressurized aerosol, the dosage unit may be determined by providing a valve to deliver a metered amount. Capsules and cartridges (e.g., composed of gelatin) for use in an inhaler or insufflator may be formulated containing a powder mix of the compound and a suitable powder base such as lactose or starch.

If the methods of the invention comprise oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pre-gelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The methods of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may in various embodiments contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

In another embodiment, the TNF-α/IL-17 DVD-Ig binding protein can be delivered in a controlled release or sustained release system, e.g., via a pump (see Langer, supra; Sefton (1987) CRC Crit. Ref. Biomed. Eng. 14:20; Buchwald et al. (1980) Surgery 88:507; Saudek et al. (1989) N. Engl. J. Med. 321:574). In another embodiment, polymeric materials can be used to achieve controlled or sustained release of the TNF-α/IL-17 DVD-Ig binding protein (see, e.g., Goodson Chapter 6, In Medical Applications of Controlled Release, Vol. II, Applications and Evaluation, (Langer and Wise, eds.) (CRC Press, Inc., Boca Raton, 1984), pp. 115-138; Ranger and Peppas (1983) J. Macromol. Sci. Rev. Macromol. Chem. 23:61; Levy et al. (1985) Science 228:190; During et al. (1989) Ann. Neurol. 25:351; Howard et al. (1989) J. Neurosurg. 7 1:105); U.S. Pat. Nos. 5,679,377; 5,916,597; 5,912,015; 5,989,463; and 5,128,326; and PCT Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In certain embodiments, the polymer used in a sustained release formulation is inert, free of leachable impurities, stable on storage, sterile, and biodegradable. In other embodiments, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of use in the practice of the invention. See, e.g., U.S. Pat. No. 4,526,938; PCT publication WO 91/05548; PCT publication WO 96/20698; Ning et al. (1996) Radiother. Oncol. 39: 179-189; Song et al. (1995) PDA J. Pharm. Sci. Tech. 50: 372-397; Cleek et al. (1997) Pro. Intl. Symp. Control. Rel. Bioact. Mater. 24: 853-854; and Lam et al. (1997) Proc. Intl. Symp. Control Rel. Bioact. Mater. 24: 759-760; each of which is incorporated herein by reference in its entirety.

In one embodiment, where the composition is a nucleic acid/nucleotide encoding the TNF-α/IL-17 DVD-Ig binding protein, the nucleic acid is prepared, by constructing it as part of an appropriate nucleic acid expression vector and administering it so that it becomes intracellular, e.g., by use of a retroviral vector (see U.S. Pat. No. 4,980,286), by direct injection, by use of microparticle bombardment (e.g., a gene gun; Biolistic, Dupont), by coating with lipids or cell-surface receptors or transfecting agents, or by administering it in linkage to a homeobox-like peptide which is known to enter the nucleus (see, e.g., Joliot et al. (1991) Proc. Natl. Acad. Sci. USA 88: 1864-1868). Alternatively, a nucleic acid can be introduced intracellularly and incorporated within host cell DNA for expression, e.g., by homologous recombination.

The TNF-α/IL-17 DVD-Ig binding protein pharmaceutical composition used in the methods of the invention is formulated to be compatible with its intended route of administration. Typically, compositions for intravenous administration are solutions in sterile isotonic aqueous buffer. The composition may also include a solubilizing agent or a local anesthetic, such as lignocaine, to ease pain at the site of the injection.

In certain embodiments the TNF-α/IL-17 DVD-Ig binding protein is linked to a half-life extending vehicle known in the art. Such vehicles include, but are not limited to, the Fc domain, polyethylene glycol, and dextran. Such vehicles are described, e.g., in U.S. Pat. No. 6,660,843 and published PCT Publication No. WO 99/25044, which are hereby incorporated by reference in their entireties for any purpose.

In various embodiments, the method further includes administering to the subject a second agent such as, for example, one or more DMARDs. In certain embodiments, the DMARD is methotrexate. In various embodiments, the DMARD is synthetic. In various embodiments, the DMARD is or comprises a biologic. In various embodiments, the DMARD is or comprises a non-biologic or small molecule. In various embodiments the DMARD is sulfasalazine, auranofin, a gold compound, an azathioprine, 6-mercaptopurine, ciclosporin A, an antimalarial agent, d-penicillamine, or a retinoid or combination thereof.

In various embodiments, administration of the DMARD is systemic or is localized to an area of the subject or diffuses to a treatment area. In various embodiments, the administration of the DMARD is intravenous or by subcutaneous injection (e.g., in the arm or abdomen).

In various embodiments, the subject has been treated with the DMARD for a period of time prior to administration of the TNF-α/IL-17 DVD-Ig binding protein. For example, the period of time is at least two days, a week or a month. In various embodiments, the period of time is about one, two, three, four, five or six months. In various embodiments, the TNF-α/IL-17 DVD-Ig binding protein is administered after administering the DMARD. In certain embodiments, the TNF-α/IL-17 DVD-Ig binding protein is administered minutes, hours, days or months after the DMARD. In various embodiments, the TNF-α/IL-17 DVD-Ig binding protein is administered approximately simultaneously with the DMARD. Alternatively, the TNF-α/IL-17 DVD-Ig binding protein is administered minutes, hours, days or months prior to administering the DMARD.

In various embodiments, administering the TNFα/IL-17 DVD-Ig binding protein improves at least one a negative condition in the subject associated with the PsA, or PsA associated symptom. In various embodiments, the PsA associated symptom is selected from the group consisting of inflammation, stiffness, pain, bone erosion, osteoporosis, joint deformity, a nerve condition, scarring, a cardiac disorder/condition, a blood vessel disorder/condition, high blood pressure, fatigue, anemia, weight loss, abnormal body temperature, fever, a lung condition/disease, a kidney condition/disorder, a liver condition/disorder, an ocular disorder/condition, a skin disorder/condition, an intestinal disorder/condition, and an infection.

Administration of the binding protein to the subject in various embodiments of the method improves a score of one or more psoriatic arthritis metrics in the subject. For example, the psoriatic arthritis metric is selected from the group consisting of: Physician Global Assessment of Disease Activity, Patient Reported Outcome, a Health Assessment Questionnaire (HAQ-DI), a patient global assessment of disease activity (VAS)), measurement or presence of an anti-drug antibody (ADA), tender joint count (TJC), swollen joint count (SJC), patient's assessment of pain, Work Instability Scale for Rheumatoid Arthritis, Short Form Health Survey (SF-36), American College of Rheumatology, ACR, (e.g., ACR20, ACR50, and ACR70), proportion of subjects achieving Low Disease Activity (LDA), Disease Activity Score 28 (DAS28, e.g., DAS28 based on C-reactive protein), Clinical Disease Activity Index (CDAI), simple disease activity index (SDAI), and Clinical Remission criteria.

This invention pertains to the administration of binding proteins, or antigen-binding portions thereof, that bind IL-17 or TNF-α, such as DVD-Ig binding proteins that bind IL-17 and TNF-α for the treatment of PsA. Various aspects of the invention relate to the use of bi-specific antibodies and antibody fragments thereof, DVD-Ig| binding proteins, and pharmaceutical compositions thereof, as well as nucleic acids, recombinant expression vectors and host cells for making such anti-IL-17/TNF-α binding proteins. The methods of the invention also encompass the use of any binding protein or antibody capable of competing with an anti-IL-17 and/or anti-TNF-α binding protein. In certain embodiments, the binding protein is a DVD-Ig binding protein comprising one or more of the sequences shown in Example 1.

### DEFINITIONS

Unless otherwise defined herein, scientific and technical terms shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. The use of “or” means “and/or” unless stated otherwise. Furthermore, the use of the term “including”, as well as other forms, such as “includes” and “included”, is not limiting.

Generally, nomenclatures and techniques used in connection with cell and tissue culture, molecular biology, immunology, microbiology, genetics and protein and nucleic acid chemistry and hybridization described herein are those well-known and commonly used in the art and are described in various general and more specific references that are cited and discussed throughout the specification unless otherwise indicated. Enzymatic reactions and purification techniques are performed according to manufacturer's specifications, as commonly performed in the art or otherwise as described herein. The nomenclatures and techniques used in connection with analytical chemistry, synthetic organic chemistry, and medicinal and pharmaceutical chemistry described herein are those well-known and commonly used in the art and are described in various general and more specific references that are cited and discussed throughout the specification unless otherwise indicated. Standard techniques are used for chemical syntheses, chemical analyses, pharmaceutical preparation, formulation, and delivery, and treatment of patients. For example, formulations and methods of producing and making compositions using a binding protein (e.g., a DVD-Ig binding protein) are described in U.S. 20140161817; and U.S. Pat. Nos. 8,835,610; and 8,779,101, each of which is incorporated by reference herein in its entirety. Select terms are defined below:

The term “dual variable domain immunoglobulin” and “DVD-Ig” means a DVD binding protein that comprises two first and two second polypeptide chains, each independently comprising VD1-(X1)n-VD2-C-(X2)n, wherein VD1 is a first variable domain; VD2 is a second variable domain; C is a constant domain; X1 is a linker; X2 is an Fc region; and n is 0 or 1. In various embodiments of the method, the DVD-Ig comprises a first and second polypeptide chains, wherein said first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; and n is independently 0 or 1; and wherein said second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1; X2 does not comprise an Fc region; and n is independently 0 or 1. A description of the design, expression, and characterization of DVD-Ig molecules is provided in PCT Publication No. WO 2007/024715, WO 2010/102251, U.S. Pat. No. 7,612,181, and Wu et al., Nature Biotech., 25: 1290-1297 (2007), incorporated herein by reference in their entireties.

The term “adalimumab” or “Humira®” means a recombinant human immunoglobulin (IgG1) monoclonal antibody containing only human peptide sequences. Adalimumab is produced by recombinant DNA technology in a mammalian cell expression system. The recombinant antibody is composed of 1330 amino acids and has a molecular weight of approximately 148 kilodaltons. Adalimumab is composed of fully human heavy and light chain variable regions, which confer specificity to human TNF, and human IgG1 heavy chain and kappa light chain sequences. Adalimumab binds with high affinity and specificity to soluble TNF-α but not to lymphotoxin-α (TNF-β). Adalimumab also modulates biological responses that are induced or regulated by TNF. After treatment with adalimumab, levels of acute phase reactants of inflammation (C-reactive protein [CRP] and erythrocyte sedimentation rate [ESR]) and serum cytokines rapidly decrease.

The term “biological activity” means all inherent biological properties of a molecule.

The terms “disease-modifying anti-rheumatic drug” and “DMARD” mean a drug or agent that modulates, reduces or treats the symptoms and/or progression associated with an immune system disease, including autoimmune diseases (e.g., rheumatic diseases and psoriatic diseases), graft-related disorders, inflammatory diseases, and immunoproliferative diseases. The DMARD may be a synthetic DMARD (e.g., a conventional synthetic disease modifying antirheumatic drug) or a biologic DMARD. For example, DMARDs include methotrexate, sulfasalazine (Azulfidine®), cyclosporine (Neoral®, Sandimmune®), leflunomide (Arava®), hydroxychloroquine (Plaquenil®), azathioprine (Imuran®), or a combination thereof. In various embodiments, a DMARD is used to treat or control progression, joint deterioration, and/or disability associated with PsA.

The term “polypeptide” means any polymeric chain of amino acids and encompasses native or artificial proteins, polypeptide analogs or variants of a protein sequence, or fragments thereof, unless otherwise contradicted by context. A polypeptide may be monomeric or polymeric. For an antigenic polypeptide, a fragment of a polypeptide optionally contains at least one contiguous or nonlinear epitope of a polypeptide. The precise boundaries of the at least one epitope fragment can be confirmed using ordinary skill in the art.

The term “variant” means a polypeptide that differs from a given polypeptide in amino acid sequence by the addition, deletion, or conservative substitution of amino acids, but that retains the biological activity of the given polypeptide (e.g., a variant TNF-α can compete with anti-TNF-α antibody for binding to TNF-α). A conservative substitution of an amino acid, i.e., replacing an amino acid with a different amino acid of similar properties (e.g., hydrophilicity and degree and distribution of charged regions) is recognized in the art as a conservative substitution. Conservative substitutions can be identified, in part, by considering the hydropathic index of amino acids, as understood in the art (see, e.g., Kyte et al. (1982) J. Mol. Biol. 157:105-132). The hydrophilicity of amino acids also can be used to identify substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydropathic index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also encompasses a polypeptide or fragment thereof that has been differentially processed, such as by proteolysis, phosphorylation, or other post-translational modification, yet retains its biological activity or antigen reactivity, e.g., the ability to bind to TNF-α and IL-17. The term “variant” encompasses fragments of a variant unless otherwise contradicted by context.

The terms “isolated protein” and “isolated polypeptide” mean a protein or polypeptide that by virtue of its origin or source of derivation is not associated with naturally associated components that accompany it in its native state; is substantially free of other proteins from the same species, is expressed by a cell from a different species, or does not occur in nature. Thus, a protein or polypeptide that is chemically synthesized or synthesized in a cellular system different from the cell from which it naturally originates will be isolated from its naturally associated components. A protein or polypeptide may also be rendered substantially free of naturally associated components by isolation using protein purification techniques well known in the art.

The terms “human IL-17” and “hIL-17” mean IL-17A. In certain embodiments, human IL-17 has the amino acid sequence of SEQ ID NO:1. IL-17A can form a homodimeric protein comprising two 15 kD IL-17A proteins (hIL-17A/A) and a heterodimeric protein comprising a 15 kD IL-17A protein and a 15 kD IL-17F protein (hIL-17A/F). The amino acid sequences of hIL-17A and hIL-17F are shown in Table 1. The term “hIL-17” includes recombinant hIL-17 (rhIL-17), which can be prepared by standard recombinant expression methods.

TABLE 1
Sequence of Human IL-17A and Human IL-17F

| | Sequence | |
|---|---|---|
| | Iden- | Sequence |
| Protein | tifier | 12345678901234567890123456789012 |
| Human | SEQ ID | GITIPRNPGCPNSEDKNFPRTVMVNLNIHNRN |
| IL-17A | NO.: 1 | TNINPKRSSDYYNRSTSPWNLHRNEDPERYPS |
| | | VIWEAKCRHLGCINADGNVDYHMNSVPIQQEI |
| | | LVLRREPPHCPNSFRLEKILVSVGCTCVTPIV |
| | | HHVA |
| Human | SEQ ID | RKIPKVGHTFFQKPESCPPVPGGSMKLDIGII |
| IL-17F | NO.: 2 | NENQRVSMSRNIESRSTSPWNYTVTWDPNRYP |
| | | SEVVQAQCRNLGCINAQGKEDISMNSVPIQQE |
| | | TLVVRRKHQGCSVSFQLEKVLVTVGCTCVTPV |
| | | IHHVQ |

The term “interleukin 17” or “IL-17” or “IL-17A” means a mammalian protein that has significant sequence homology to “human IL-17” and “hIL-17”. According to certain embodiments, that significant sequence homology is at least 70, 75, 80, 85, 90, 95, 96, 97, 98, or 99%.

The term “IL-17/TNF-α binding protein” means a bispecific binding protein (e.g., DVD-Ig protein) that binds IL-17 and TNF-α. The relative positions of the TNF-α binding region and IL-17 binding region within the bispecific binding protein are not fixed (e.g., VD1 or VD2 of the DVD-Ig protein) unless specifically specified herein.

The terms “human TNF-α,” “hTNF-α,” and “hTNF” mean a 17 kD secreted form and a 26 kD membrane associated form of a human cytokine, the biologically active form of which is composed of a trimer of noncovalently bound 17 kD molecules. The structure of hTNF-α is described further in, for example, Pennica et al. (1984) Nature 312:724-729; Davis et al. (1987) Biochem. 26:1322-1326; and Jones et al. (1989) Nature 338:225-228. The term “hTNF-α” includes recombinant human TNF-α (“rhTNF-α”). The amino acid sequence of hTNF-α is shown in Table 2.

TABLE 2
Sequence of Human TNF-α

| | Sequence | |
|---|---|---|
| | Iden- | Sequence |
| Protein | tifier | 12345678901234567890123456789012 |
| Human | SEQ ID | MSTESMIRDVELAEEALPKKTGGPQGSRRCLF |
| TNF-a | NO.: 3 | LSLFSFLIVAGATTLFCLLHFGVIGPQREEFP |
| | | RDLSLISPLAQAVRSSSRTPSDKPVAHVVANP |
| | | QAEGQLQWLNRRANALLANGVELRDNQLVVPS |
| | | EGLYLIYSQVLFKGQGCPSTHVLLTHTISRIA |
| | | VSYQTKVNLLSAIKSPCQRETPEGAEAKPWYE |
| | | PIYLGGVFQLEKGDRLSAEINRPDYLDFAESG |
| | | QVYFGIIAL |

The term “tumor necrosis factor,” “TNF”, “TNFα” and “TNF-α” means a mammalian protein that has significant sequence homology to “human TNF-α,” “hTNF-α,” and “hTNF”. According to certain embodiments, that significant sequence homology is at least 70, 75, 80, 85, 90, 95, 96, 97, 98, or 99%.

The terms “specific binding” or “specifically binding,” in reference to the interaction of an antibody, a protein, or a peptide with a second chemical species, mean that the interaction is dependent upon the presence of a particular structure (e.g., an antigenic determinant or epitope) on the chemical species. If an antibody is specific for epitope “A”, in the presence of a molecule containing epitope A (or free, unlabeled epitope A) in which “A” is labeled, the antibody will reduce the amount of labeled A bound to the antibody.

“Specific binding partner” is a member of a specific binding pair. The term “specific binding pair” comprises two different molecules, which specifically bind to each other through chemical or physical means (e.g., an antigen (or fragment thereof) and an antibody (or antigenically reactive fragment thereof)). Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced. The terms “specific” and “specificity” in the context of an interaction between members of a specific binding pair refer to the selective reactivity of the interaction.

The term “antibody” means any immunoglobulin (Ig) molecule comprised of four polypeptide chains, two heavy (H) chains and two light (L) chains, or any functional fragment, mutant, variant, or derivative thereof, which retains the essential epitope binding features of an Ig molecule. Such mutant, variant, or derivative antibody formats are known in the art, non-limiting embodiments of which are discussed below.

The term “human antibody” includes antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” means human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “CDR” means the complementarity determining region within antibody variable sequences. There are three CDRs in each of the variable regions of the heavy chain and the light chain, which are designated CDR1, CDR2, and CDR3, for each of the variable regions. The term “CDR set” means a group of three CDRs that occur in a single variable region (i.e., VH or VL) of an antigen binding site. The exact boundaries of these CDRs have been defined differently according to different systems. The system described by Kabat (Kabat et al. (1987, 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196: 901-917 and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3, where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139 and MacCallum (1996) J. Mol. Biol. 262(5): 732-745). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The terms “Kabat numbering,” “Kabat definition,” and “Kabat labeling” mean a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190: 382-391 and Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The growth and analysis of extensive public databases of amino acid sequences of variable heavy and light regions over the past twenty years have led to the understanding of the typical boundaries between framework regions (FR) and CDR sequences within variable region sequences and enabled persons skilled in this art to accurately determine the CDRs according to Kabat numbering, Chothia numbering, or other systems. See, e.g., Martin, “Protein Sequence and Structure Analysis of Antibody Variable Domains,” In Kontermann and Dübel, eds., Antibody Engineering (Springer-Verlag, Berlin, 2001), chapter 31, pages 432-433. A useful method of determining the amino acid sequences of Kabat CDRs within the amino acid sequences of variable heavy (VH) and variable light (VL) regions is provided below:

To identify a CDR-L1 amino acid sequence:
Starts approximately 24 amino acid residues from the amino terminus of the VL region;
Residue before the CDR-L1 sequence is always cysteine (C);
Residue after the CDR-L1 sequence is always a tryptophan (W) residue, typically Trp-Tyr-Gln (W-Y-Q), but also Trp-Leu-Gln (W-L-Q), Trp-Phe-Gln (W-F-Q), and Trp-Tyr-Leu (W-Y-L);
Length is typically 10 to 17 amino acid residues.

To identify a CDR-L2 amino acid sequence:
Starts always 16 residues after the end of CDR-L1;
Residues before the CDR-L2 sequence are generally Ile-Tyr (I-Y), but also Val-Tyr (V-Y), Ile-Lys (I-K), and Ile-Phe (I-F);
Length is always 7 amino acid residues.

To identify a CDR-L3 amino acid sequence:
Starts always 33 amino acids after the end of CDR-L2;
Residue before the CDR-L3 amino acid sequence is always a cysteine (C);
Residues after the CDR-L3 sequence are always Phe-Gly-X-Gly (F-G-X-G) (SEQ ID NO:7), where X is any amino acid;
Length is typically 7 to 11 amino acid residues.

To identify a CDR-H1 amino acid sequence:
Starts approximately 31 amino acid residues from amino terminus of VH region and always 9 residues after a cysteine (C);
Residues before the CDR-H1 sequence are always Cys-X-X-X-X-X-X-X-X (SEQ ID NO:10), where X is any amino acid;
Residue after CDR-H1 sequence is always a Trp (W), typically Trp-Val (W-V), but also Trp-Ile (W-I), and Trp-Ala (W-A);
Length is typically 5 to 7 amino acid residues.

To identify a CDR-H2 amino acid sequence:
Starts always 15 amino acid residues after the end of CDR-H1;
Residues before CDR-H2 sequence are typically Leu-Glu-Trp-Ile-Gly (L-E-W-I-G) (SEQ ID NO:8), but other variations also;
Residues after CDR-H2 sequence are Lys/Arg-Leu/Ile/Val/Phe/Thr/Ala-Thr/Ser/Ile/Ala (K/R-L/I/V/F/T/A-T/S/I/A);
Length is typically 16 to 19 amino acid residues.

To identify a CDR-H3 amino acid sequence:
Starts always 33 amino acid residues after the end of CDR-H2 and always 3 after a cysteine (C)′
Residues before the CDR-H3 sequence are always Cys-X-X (C-X-X), where X is any amino acid, typically Cys-Ala-Arg (C-A-R);
Residues after the CDR-H3 sequence are always Trp-Gly-X-Gly (W-G-X-G) (SEQ ID NO:9), where X is any amino acid;
Length is typically 3 to 25 amino acid residues.

With respect to constructing DVD-Ig binding protein or other binding protein molecules, the term “linker”, “peptide linker”, or “linker polypeptide” means a single amino acid or a polypeptide comprising two or more amino acid residues joined by peptide bonds used to link one or more antigen binding portions. Such linker polypeptides are well known in the art (see, e.g., Holliger et al., (1993) Proc. Natl. Acad. Sci. USA, 90: 6444-6448; Poljak (1994) Structure, 2: 1121-1123). Exemplary linkers include, but are not limited to, GGGGSG (SEQ ID NO:14), GGSGG (SEQ ID NO:15), GGGGSGGGGS (SEQ ID NO:16), GGSGGGGSG (SEQ ID NO:17), GGSGGGGSGS (SEQ ID NO:18), GGSGGGGSGGGGS (SEQ ID NO:19), GGGGSGGGGSGGGG (SEQ ID NO:20), GGGGSGGGGSGGGGS (SEQ ID NO:21), ASTKGP (SEQ ID NO:22), ASTKGPSVFPLAP (SEQ ID NO:23), TVAAP (SEQ ID NO:24), RTVAAP (SEQ ID NO:25), TVAAPSVFIFPP (SEQ ID NO:26), RTVAAPSVFIFPP (SEQ ID NO:27), AKTTPKLEEGEFSEAR (SEQ ID NO:28), AKTTPKLEEGEFSEARV (SEQ ID NO:29), AKTTPKLGG (SEQ ID NO:30), SAKTTPKLGG (SEQ ID NO:31), SAKTTP (SEQ ID NO:32), RADAAP (SEQ ID NO:33), RADAAPTVS (SEQ ID NO:34), RADAAAAGGPGS (SEQ ID NO:35), RADAAAAGGGGSGGGGSGGGGSGGGGS (SEQ ID NO:36), SAKTTPKLEEGEFSEARV (SEQ ID NO:37), ADAAP (SEQ ID NO:38), ADAAPTVSIFPP (SEQ ID NO:39), QPKAAP (SEQ ID NO:40), QPKAAPSVTLFPP (SEQ ID NO:41), AKTTPP (SEQ ID NO:42), AKTTPPSVTPLAP (SEQ ID NO:43), AKTTAP (SEQ ID NO:44), AKTTAPSVYPLAP (SEQ ID NO:45), GENKVEYAPALMALS (SEQ ID NO:46), GPAKELTPLKEAKVS (SEQ ID NO:47), and GHEAAAVMQVQYPAS (SEQ ID NO:48).

The term “neutralizing” means to reduce activity, e.g., the biological activity of an antigen (e.g., the cytokines TNF-α and IL-17) when a binding protein specifically binds the antigen. Preferably, a neutralizing binding protein described herein binds to human TNF-α and/or human IL-17 resulting in the inhibition of a biological activity of the cytokines. Preferably, the neutralizing binding protein binds TNF-α and IL-17 and reduces a biologically activity of TNF-α and IL-17 by at least about 20%, 30%, 40%, 50%, 60%, 70%, 80%, 85%, 90%, 95%, or more. Inhibition of a biological activity of TNF-α and IL-17 by a neutralizing binding protein can be assessed by measuring one or more indicators of TNF-α and IL-17 biological activity well known in the art.

The term “activity” includes activities such as the binding specificity/affinity of an antibody for an antigen, for example, an anti-TNF-α and/or anti-IL-17 antibody that binds to TNF-α and/or IL-17.

The term “epitope” means a polypeptide determinant capable of specific binding to an immunoglobulin or T-cell receptor. In certain embodiments, epitope determinants include chemically active surface groupings of molecules such as amino acids, sugar side chains, phosphoryl or sulfonyl groups, and, in certain embodiments, may have specific three dimensional structural characteristics and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Antibodies are said to bind to the same epitope if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “percent identity” means a quantitative measurement of the similarity between two sequences (complete amino acid sequence or a portion thereof). Calculations of sequence identity between sequences are known by those in the art. For example, to determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences. For example, percent identity between two amino acid or nucleic acid sequences can be about 30%, 35%, 40%, 45%, 50%, 55%, 60%, 65%, 70%, 75%, 80%, 85%, 90%, 95%, 96%, 98%, 99%, or 99% or more.

The comparison of sequences and determination of percent identity between two sequences are accomplished using a mathematical algorithm. Percent identity between two amino acid sequences may be determined using an alignment software program using the default parameters. Suitable programs include, for example, CLUSTAL W (see Thompson et al. (1994) Nucl. Acids Res. 22: 4673-4680) or CLUSTAL X.

The term “substantially identical” in reference to amino acid sequences means a first amino acid sequence that contains a sufficient or minimum number of amino acid residues that are identical to aligned amino acid residues in a second amino acid sequence such that the first and second amino acid sequences can have a common structural domain and/or common functional activity. For example, a protein with an amino acid sequence that contain a common structural domain having at least about 30%, 35%, 40%, 45%, 50%, 55%, 60%, 65%, 70%, 75%, 80%, 85%, 90%, 95%, 96%, 98%, 99%, or 99% or more identity to a DVD-Ig binding protein described herein (e.g., a DVD-Ig binding protein comprising SEQ ID NO: 4, SEQ ID NO: 9, or a portion or combination thereof) would be substantially identical to that DVD-Ig binding protein. In various embodiments, the substantially identical protein includes an amino acid sequence that is at least about 30%, about 35%, about 40%, about 45%, about 50%, about 55%, about 60%, about 65%, about 70%, about 75%, about 80%, about 85%, about 90%, about 95%, about 99%, or 99% or more identical to SEQ ID NO: 4, SEQ ID NO: 9, or a portion or a combination thereof.

The term “surface plasmon resonance” is an optical detection process that allows for the analysis of real-time molecular interactions (for example bispecific interactions and binding constants) by detection of alterations in protein concentrations within a biosensor matrix, for example using the BIAcore system (Pharmacia Biosensor AB, Uppsala, Sweden and Piscataway, N.J.). For further descriptions, see Jönsson et al. (1993) Ann. Biol. Clin. 51: 19-26; Jönsson et al. (1991), BioTechniques 11: 620-627; Johnsson et al. (1995) J. Mol. Recognit. 8: 125-131; and Johnsson et al. (1991) Anal. Biochem. 198: 268-277.

The terms “Kₒₙ,” “Kon,” and “kon” refer to the on rate constant for association or “association rate constant,” of a binding protein (e.g., an antibody) to an antigen to form an association complex, e.g., antibody/antigen complex, as is known in the art. The term “Kₒₙ” also is known by the terms “association rate constant” or “ka”. This value indicates the binding rate of an antibody to its target antigen or the rate of complex formation between an antibody and antigen as is shown by the equation below:

Antibody (“Ab”)+Antigen (“Ag”)→Ab−Ag

The terms “K_{off},” “Koff,” and “koff” refer to the off rate constant for dissociation, or “dissociation rate constant,” of a binding protein (e.g., an antibody) from an association complex (e.g., an antibody/antigen complex) as is known in the art. This value indicates the dissociation rate of an antibody from its target antigen or separation of Ab−Ag complex over time into free antibody and antigen as shown by the equation below:

Ab+Ag←Ab−Ag

The terms “K_{D}” and “K_{d}” refer to the “equilibrium dissociation constant,” and refers to the value obtained in a titration measurement at equilibrium, or by dividing the dissociation rate constant (Koff) by the association rate constant (Kon). The association rate constant (Kon), the dissociation rate constant (Koff), and the equilibrium dissociation constant (K are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used. Additionally, a KinExA® (Kinetic Exclusion Assay) assay, available from Sapidyne Instruments (Boise, Id.) can also be used.

The terms “AUC” and “area under the curve” refer to the area under the plasma drug concentration-time curve and reflects the actual body exposure to drug after administration of a dose of the drug. AUC is typically related to clearance. In various embodiments, a higher clearance may be related to a smaller AUC, and a lower clearance rate may be related to a larger AUC value. The AUC higher values may in various embodiments represent slower clearance rates.

The term “volume of distribution” means the theoretical volume of fluid into which the total drug administered would have to be diluted to produce the concentration in plasma. Calculating the volume of distribution may in various embodiments involve the quantification of the distribution of a drug, e.g., a TNF-α/IL-17 DVD-Ig binding protein, or antigen-binding portion thereof, between plasma and the rest of the body after dosing. The volume of distribution is the theoretical volume in which the total amount of drug would need to be uniformly distributed in order to produce the desired blood concentration of the drug.

The terms “half-life” and “T½” mean the period of time for half of a drug's concentration or activity (e.g., pharmacologic or physiologic) to be reduced by one-half. For example, the half-life may involve the time taken for half of the dose to be eliminated, excreted or metabolized.

The term “Cmax” means the peak concentration that a drug is observed, quantified or measured in a specified fluid or sample after the drug has been administered. In various embodiments, determining the Cmax involves in part quantification of the maximum or peak serum or plasma concentration of a drug/therapeutic agent observed in a sample from a subject administered the drug.

The term “bioavailability” means the degree to which a drug is absorbed or becomes available to cells or tissue after administration of the drug. For example, bioavailability in certain embodiments involves quantification of the fraction or percent of a dose which is absorbed and enters the systemic circulation after administration of a given dosage form. See international publication number WO2013078135 published May 30, 2013, which is incorporated by reference herein in its entirety.

The terms “label” and “detectable label” mean a moiety attached to a specific binding partner, such as an antibody or an analyte, e.g., to render the reaction between two specific binding partners detectable. The specific binding partner so labeled is referred to as “detectably labeled”. Thus, the term “labeled binding protein” means a protein with a label incorporated that provides for the identification of the binding protein or the ligand to which it binds. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin or streptavidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm), chromogens, fluorescent labels (e.g., FITC, rhodamine, lanthanide phosphors), enzymatic labels (e.g., horseradish peroxidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), and magnetic agents (e.g., gadolinium chelates). Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety. Use of the term “detectably labeled” is intended to encompass the latter type of detectable labeling.

The term “binding protein conjugate” means a binding protein that is chemically linked to a second chemical moiety, such as a therapeutic or cytotoxic agent.

The term “agent” means a chemical compound, a mixture of chemical compounds, a biological molecule (e.g., a biological macromolecule), or an extract made from biological materials. When employed in the context of an immunoassay, a binding protein conjugate may be a detectably labeled antibody, which is used as the detection antibody.

The terms “crystal” and “crystallized” mean an agent in the form of a crystal. Crystals are one form of the solid state of matter that is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. See Giegé et al., Chapter 1, In Crystallization of Nucleic Acids and Proteins, a Practical Approach, 2nd ed., (Ducruix and Giegé, eds.) (Oxford University Press, New York, 1999) pp. 1-16.

The term “polynucleotide” means a polymer of two or more nucleotides, e.g., ribonucleotides or deoxynucleotides or a modified form of nucleotide. The term includes single and double stranded forms of DNA.

The term “isolated polynucleotide” means a polynucleotide (e.g., of genomic, cDNA, or synthetic origin, or some combination thereof) that, by virtue of its origin, is not associated with all or a portion of a polynucleotide with which the polynucleotide is found in nature; is operably linked to a polynucleotide that it is not linked to in nature; or does not occur in nature as part of a larger sequence.

The term “vector” means a nucleic acid molecule capable of transporting another nucleic acid to which it has been linked into a cell, where it can be replicated and/or expressed. One type of vector is a “plasmid”, which refers to a circular double stranded DNA loop into which additional DNA segments may be ligated. Another type of vector is a viral vector, wherein additional nucleic acid segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked (“recombinant expression vectors” or “expression vectors”). In general, expression vectors are often in the form of plasmids. Vectors may also be viral vectors (e.g., replication defective retroviruses, adenoviruses and adeno-associated viruses).

The term “operably linked” refers to a juxtaposition wherein the components described are in a relationship permitting them to function in their intended manner. A control sequence that is “operably linked” to a coding sequence is ligated in such a way that expression of the coding sequence is achieved under conditions compatible with the control sequences. Operably linked sequences include both expression control sequences that are contiguous with the gene of interest and expression control sequences that act in trans or at a distance to control the gene of interest. The term “expression control sequence” means a polynucleotide sequence that is necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequence” means a sequence whose presence is essential for expression and processing, and can also include additional components whose presence is advantageous, for example, leader sequences and fusion partner sequences.

The term “transformation” means a process by which exogenous DNA enters a host cell. Transformation may occur under natural or artificial conditions using various methods well known in the art. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

The terms “recombinant host cell” and “host cell” mean a cell into which exogenous DNA has been introduced. In an embodiment, the host cell comprises two or more (e.g., multiple) nucleic acids encoding antibodies. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term host cell. In an embodiment, host cells include prokaryotic and eukaryotic cells selected from any of the kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, COS, NS0, SP2 and PER.C6; the insect cell line Sf9; and the fungal cell Saccharomyces cerevisiae. In various embodiments, the host cells are non-human host cells.

Standard techniques may be used for recombinant DNA, oligonucleotide synthesis, tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N.Y., 1989).

The term “modulator” means a compound capable of changing or altering an activity or function of a molecule of interest (e.g., the biological activity of hTNF-α and hIL-17). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described in various publications, e.g., in PCT Publication No. WO2001/83525, incorporated by reference herein in its entirety.

The term “agonist” means a modulator that, when contacted with a molecule of interest, causes an increase in the magnitude of a certain activity or function of the molecule compared to the magnitude of the activity or function observed in the absence of the agonist. Particular agonists of interest may include, but are not limited to, TNF-α and IL-17 polypeptides, nucleic acids, carbohydrates, or any other molecule that binds to hTNF-α and hIL-17.

The terms “antagonist” and “inhibitor” mean a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human TNF-α and IL-17. Antagonists and inhibitors of human TNF-α and IL-17 may include, but are not limited to, proteins, nucleic acids, carbohydrates, or any other molecules, which bind to human TNF-α and IL-17.

The term “effective amount” means the amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one or more symptoms associated with a disorder; detect a disorder; or enhance or improve the prophylactic or therapeutic effect(s) of another therapy (e.g., prophylactic or therapeutic agent).

The terms “patient” and “subject” mean an animal, such as a mammal, including a primate (for example, a human, a monkey, and a chimpanzee), a non-primate (for example, a cow, a pig, a camel, a llama, a horse, a goat, a rabbit, a sheep, a hamster, a guinea pig, a cat, a dog, a rat, a mouse, a whale), a bird and a fish. In an embodiment, the patient or subject is a human, such as a human being treated or assessed for a disease, disorder or condition; a human at risk for a disease, disorder or condition; and/or a human having a disease, disorder or condition.

The term “sample” means a quantity of a substance. The term “biological sample” means a quantity of a substance obtained from a living thing or a formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “component” means a portion of a mixture, composition, system or kit, for example a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), an analyte, a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

The term “control” means a component or composition that is not, or does not contain, an analyte (“negative control”) or is or contains analyte (“positive control”). A positive control can comprise a known concentration of analyte. A “calibrator” means a composition comprising a known concentration of analyte. A positive control can be used to establish assay performance characteristics and is a useful indicator of the integrity of reagents (e.g., analytes).

The terms “predetermined cutoff” and “predetermined level” mean an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, cutoff values may vary depending on the nature of the immunoassay (e.g., antibodies employed). It is well within the ordinary skill of one in the art to adapt the disclosure herein for other immunoassays to obtain immunoassay-specific cutoff values for those other immunoassays. Whereas the precise value of the predetermined cutoff/level may vary between assays, correlations as described herein (if any) should be generally applicable.

The term “risk” means the possibility or probability of a particular event occurring either presently or at some point in the future. The term “risk stratification” means an array of known clinical risk factors that allows physicians to classify patients into a low, moderate, high or highest risk of developing a particular disease, disorder or condition.

The terms “DMARD resistance” and “resistance to a DMARD” means an observed or demonstrated loss of efficacy over time to treatment of a disease, disorder or condition (e.g., PsA) using a DMARD. DMARD resistance may be a multifactorial event including enhanced drug efflux via ABC transporters, impaired drug uptake and drug activation, enhanced drug detoxification etc. In various embodiments, the subject is observed to have a PsA symptom that is not reduced by DMARD treatment.

A number of abbreviations are used herein to describe aspects of the invention. Below is a list of commonly used abbreviations.
ACR American College of Rheumatology
ADA Anti-drug antibody
AE Adverse event
ALT Alanine aminotransferase
ANC Absolute neutrophil count
AS Ankylosing Spondylitis
AST Aspartate aminotransferase
BASDAI Bath ankylosing spondylitis disease activity index
BCG Bacillus Calmette-Guérin
BL Baseline
BUN Blood Urea Nitrogen
CASPAR Classification criteria for Psoriatic Arthritis
C1M Matrix metalloproteinase-mediated degradation of type I collagen
C2M Matrix metalloproteinase-mediated degradation of type II collagen
C3M Matrix metalloproteinase-mediated degradation of type III collagen
CBC Complete blood count
COX Cyclooxygenase
CRA Clinical research associate
CRPM Matrix metalloproteinase-mediated C-reactive protein
CRO Contract research organization
csDMARD Conventional synthetic disease modifying antirheumatic drugs
CTX-II C-terminal telopeptide type II collagen
CXR Chest x-ray
DAS28 Disease activity score 28
DMARD Disease modifying antirheumatic drugs
DNA Deoxyribonucleic acid
DR Disease Response
DVD-Ig Dual variable domain immunoglobulin
ECG Electrocardiogram
eCRF Electronic case report form
EDC Electronic data capture
EOW Every Other Week
ESRB External Safety Review Board
ET Early Termination
EW Every Week
FDA US Food and Drug Administration
FU Follow-Up
GCP Good Clinical Practice
HAQ-S Health Assessment Questionnaire Modified for the Spondyloarthropathies
HbcAb Hepatitis B core antibody
HbsAb Antibody to Hepatitis B surface antigen
HBsAg Hepatitis B surface antigen
HBV Hepatitis B virus
HCV Hepatitis C virus
HIV Human Immunodeficiency Virus
hrs Hours
hsCRP High sensitivity C-reactive protein
ICF Informed consent form
ICH International Conference on Harmonization
IEC Independent Ethics Committee
IGRA Interferon gamma release assay
IL Interleukin
IRB Institutional Review Board
ISR Injection site reaction
IUD Intrauterine Device
IV Intravenous
IVRS Interactive voice response system
IWRS Interactive web response system
JAK Janus kinase
JIA Juvenile idiopathic arthritis
mAB monoclonal antibody
MAD Multiple ascending dose
MDA Minimal disease activity
MedDRA Medical Dictionary for Regulatory Activities
MMP-3 Matrix metalloproteinase 3
mRNA Messenger ribonucleic acid
MTX Methotrexate
NRS Numeric rating scale
NSAID Nonsteroidal anti-inflammatory drugs
NYHA New York Heart Association
OLE Open-Label Extension
PASDAS Psoriatic disease activity score
PASI Psoriasis Area and Severity Index
PBMC Peripheral blood mononuclear cells
PCR Polymerase Chain Reaction
PD Premature Discontinuation or Pharmacodynamic
PDE4 Phosphodiesterase type 4 inhibitor
PFS Pre-filled syringe
PG Pharmacogenetic
PGA Physician's Global Assessment of Disease Activity
PK Pharmacokinetic
POR Proof of receipt
PPD Purified Protein Derivative
Ps Psoriasis
PsA Psoriatic arthritis
PT Preferred term
PtGA Patient's Global Assessment of Disease Activity
PUVA Psoralen and ultraviolet A
RA Rheumatoid Arthritis
RBC Red blood cells
RNA Ribonucleic acid
SAE Serious adverse event
SAPS Self-Assessment of Psoriasis Symptoms
SC Subcutaneous
SCR Screening
SDP Study designated physician
SF-36v2 Short form health survey
SJC Swollen joint count
SOC System organ class
SPARCC Spondyloarthritis Research Consortium of Canada
SUSAR Suspected unexpected serious adverse reactions
TB Tuberculosis
TJC Tender joint count
TNF Tumor necrosis factor
UC Ulcerative colitis
ULN Upper limit of normal
UVA Ultraviolet A
VAS Visual analog scale
VICM MMP generated citrullinated vimentin fragment
WBC White blood cell
WHO World Health Organization

### Pharmacokinetic and Statistical Abbreviations

ANCOVA Analysis of covariance
AUC Area under the curve
AUC∞ Area under the curve from time zero to infinity
AUCt Area under curve from time zero to time t
C Concentration
Ct Concentration at a specified time t after the administration of a dose
CL/F Apparent clearance
Cmax Maximum observed plasma concentration
Ctrough Observed serum concentration at the end of dosing interval
ECDF Empirical cumulative distribution function
F Bioavailability
ITT Intent-to-treat
KS Kolmogorov-Smirnov
LLQ Lower limit of quantification
LOCF Last observation carried forward
IR Insufficient responder
MAT Mean absorption time
NR Non-responder
NRI Non-responder imputation
OC Observed cases
pKa Acid dissociation constant at logarithmic scale
Rac Accumulation ratio
Rac(AUC) Accumulation ratio calculated from AUCτ,ss and AUCτ after single dosing
t½ half-life
t½abs Absorption half-life
Tmax Time to maximum observed plasma concentration
ULQ Upper limit of quantification
V/F Apparent volume of distribution

### Anaphylaxis Indicators

1. Acute onset of an illness (minutes to several hours) with involvement of the skin, mucosal tissue, or both (e.g., generalized hives, pruritus or flushing, swollen lips-tongue-uvula) and at least one of the following:
a. Respiratory compromise (e.g., dyspnea, wheeze bronchospasm, stridor, reduced peak expiratory flow, hypoxemia).
b. Reduced BP or associated symptoms or end-organ dysfunction (e.g., hypotonia [collapse], syncope, incontinence).
2. Two or more of the following that occur within minutes to several hours to study drug.
a. Involvement of the skin-mucosal tissue (e.g., generalized hives, itch-flush, swollen lips tongue-uvula).
b. Respiratory compromise (e.g., dyspnea, wheeze-bronchospasm, stridor, reduced PEF, hypoxemia).
c. Reduced BP or associated symptoms (e.g., crampy abdominal pain, vomiting).
d. Persistent gastrointestinal symptoms (e.g., crampy abdominal pain, vomiting).
3. Reduced BP after exposure to study drug (within minutes to several hours), with systolic BP of less than 90 mmHg or greater than 30% decrease from that person's baseline.* * Sampson et al. (2006). J Allergy Clin Immunol. 117(2):391-7.

### Serious Systemic Hypersensitivity Reaction

A hypersensitivity reaction is a clinical sign or symptom, or constellation of signs or symptoms, caused by an inappropriate and excessive immunologic reaction to study drug administration. A systemic hypersensitivity reaction is a hypersensitivity reaction that does not occur at the local site of study drug administration (e.g., not an injection site reaction). A serious systemic hypersensitivity reaction is a systemic hypersensitivity reaction that fulfills criteria for a serious adverse event.

### CASPAR Classification Criteria

To meet the CASPAR (Classification Criteria for Psoriatic Arthritis) criteria, * a patient must have inflammatory articular disease (joint, spine, or entheseal) with >3 points from the following 5 categories: * The CASPAR criteria have specificity of 98.7% and sensitivity of 91.4%.
1. Evidence of current psoriasis, a personal history of psoriasis, or a family history of psoriasis. Current psoriasis is defined as psoriatic skin or scalp disease present today as judged by a rheumatologist or dermatologist.† † Current psoriasis is assigned a score of 2; all other features are assigned a score of 1.
A personal history of psoriasis is defined as a history of psoriasis that may be obtained from a patient, family physician, dermatologist, rheumatologist, or other qualified healthcare provider. A family history of psoriasis is defined as a history of psoriasis in a first- or second-degree relative according to patient report.
2. Typical psoriatic nail dystrophy including onycholysis, pitting, and hyperkeratosis observed on current physical examination.
3. A negative test result for the presence of rheumatoid factor by any method except latex but preferably by enzyme-linked immuosorbent assay or nephelometry, according to the local laboratory reference range.
4. Either current dactylitis, defined as swelling of an entire digit, or a history of dactylitis recorded by a rheumatologist.
5. Radiographic evidence of juxtaarticular new bone formation, appearing as ill-defined ossification near joint margins (but excluding osteophyte formation) on plain radiographs of the hand or foot.
   Taylor et al. (2006) Arthr. Rheum. 54(8): 2665-73.

It will be readily apparent to those skilled in the art that other suitable modifications and adaptations of the methods of the invention described herein are obvious and may be made using suitable equivalents without departing from the scope of the invention or the embodiments disclosed herein.

The invention will be more clearly understood by reference to the following examples, which are included for purposes of illustration only and are not intended to be limiting.

### EXEMPLIFICATION

### Example 1

### Construction of TNF/IL-17 DVD-Ig Binding Proteins

Numerous human anti-human TNF/IL-17 dual variable domain immunoglobulin (DVD-Ig) proteins were constructed. The dual binding and/or neutralization of TNF-α and IL-17 may provide superior efficacy to the current standard of care treatments for psoriatic arthritis and other inflammatory diseases described herein. Shown below are amino acid sequences of TNF and IL-17 DVD-Ig binding proteins including the heavy and light chain amino acid sequences of ABT-122.

TABLE 3
DVD-Ig Protein Heavy Variable Domain and
Light Variable Domain of an Anti-IL-17/
TNF DVD-Ig Protein Used in the Study

| | | |
|---|---|---|
| DVD HEAVY | SEQ ID NO.: 4 | EVQLVESGGGLVQPGRSLRL |
| VARIABLE | | SCAASGFTFDDYAMHWVRQA |
| D2E7-GS10-B6-17 | | PGKGLEWVSAITWNSGHIDY |
| DVD-Ig Protein | | ADSVEGRFTISRDNAKNSLY |
| | | LQMNSLRAEDTAVYYCAKVS |
| | | YLSTASSLDYWGQGTLVTVS |
| | | SGGGGSGGGGSEVQLVQSGA |
| | | EVKKPGSSVKVSCKASGGSF |
| | | GGYGIGWVRQAPGQGLEWMG |
| | | GITPFFGFADYAQKFQGRVT |
| | | ITADESTTTAYMELSGLTSD |
| | | DTAVYYCARDPNEFWNGYYS |
| | | THDFDSWGQGTTVTVSS |
| D2E7 VH | SEQ ID NO.: 5 | EVQLVESGGGLVQPGRSLRL |
| | | SCAASGFTFDDYAMHWVRQA |
| | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | S |
| LINKER | SEQ ID NO.: 6 | GGGGSGGGGS |
| B6-17 VH | SEQ ID NO.: 7 | EVQLVQSGAEVKKPGSSVKV |
| | | SCKASGGSFGGYGIGWVRQA |
| | | PGQGLEWMGGITPFFGFADY |
| | | AQKFQGRVTITADESTTTAY |
| | | MELSGLTSDDTAVYYCARDP |
| | | NEFWNGYYSTHDFDSWGQGT |
| | | TVTVSS |
| CH | SEQ ID NO.: 8 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| DVD LIGHT | SEQ ID NO.: 9 | DIQMTQSPSSLSASVGDRVT |
| VARIABLE | | ITCRASQGIRNYLAWYQQKP |
| D2E7-GS10-B6-17 | | GKAPKLLIYAASTLQSGVPS |
| DVD-Ig Protein | | RFSGSGSGTDFTLTISSLQP |
| | | EDVATYYCQRYNRAPYTFGQ |
| | | GTKVEIKRGGSGGGGSGEIV |
| | | LTQSPDFQSVTPKEKVTITC |
| | | RASQDIGSELHWYQQKPDQP |
| | | PKLLIKYASHSTSGVPSRFS |
| | | GSGSGTDFTLTINGLEAEDA |
| | | GTYYCHQTDSLPYTFGPGTK |
| | | VDIKR |
| D2E7 VL | SEQ ID NO.: 10 | DIQMTQSPSSLSASVGDRVT |
| | | ITCRASQGIRNYLAWYQQKP |
| | | GKAPKLLIYAASTLQSGVPS |
| | | RFSGSGSGTDFTLTISSLQP |
| | | EDVATYYCQRYNRAPYTFGQ |
| | | GTKVEIKR |
| LINKER | SEQ ID NO.: 11 | GGSGGGGSG* |
| B6-17 VL | SEQ ID NO.: 12 | EIVLTQSPDFQSVTPKEKVT |
| | | ITCRASQDIGSELHWYQQKP |
| | | DQPPKLLIKYASHSTSGVPS |
| | | RFSGSGSGTDFTLTINGLEA |
| | | EDAGTYYCHQTDSLPYTFGP |
| | | GTKVDIKR |
| CL | SEQ ID NO.: 13 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

*Note that in some embodiments, the C-terminus includes a serine (i.e., the light chain linker amino acid is GGSGGGGSGS (SEQ ID NO: 18))

### Example 2

### Preliminary Studies of Patients Administered ABT-122

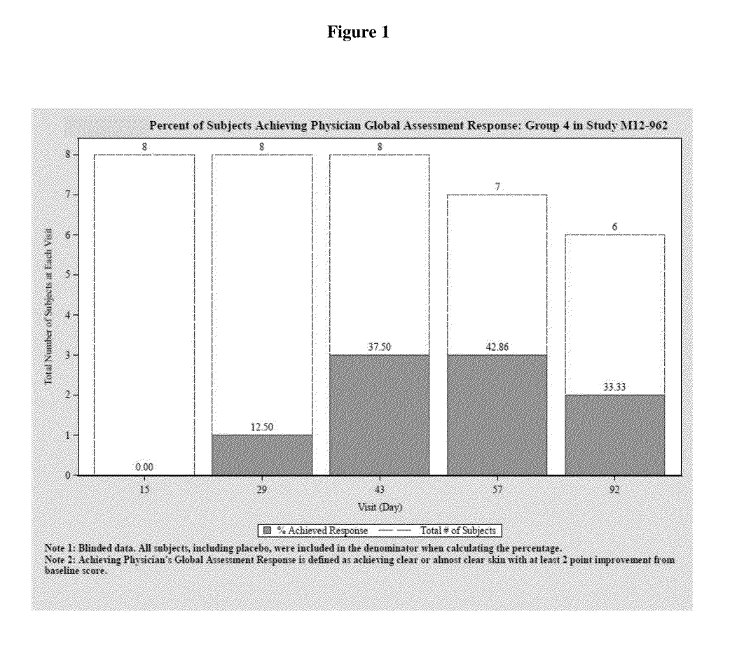

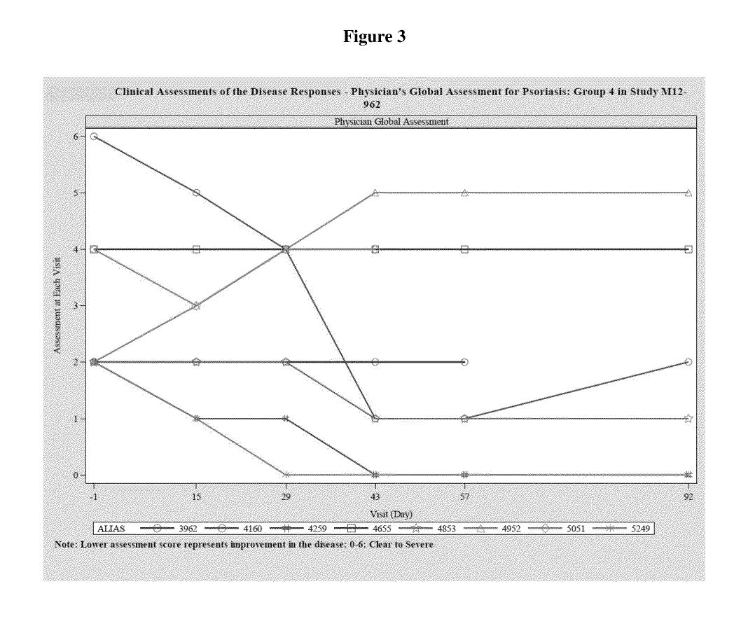

Data in FIG. 1 show that at visit days 29, 43, 57 and 92 the percent of subjects achieving Physician Global Assessment response was 12.50%, 37.50%, 42.86%, and 33.33%. In each group of about 6-8 subjects for each visit there were two placebo subjects which artificially reduced the therapeutic percentages for treatment with ABT-122 DVD-Ig binding protein. FIG. 3 shows individual subject scores for Physician Global Assessment.

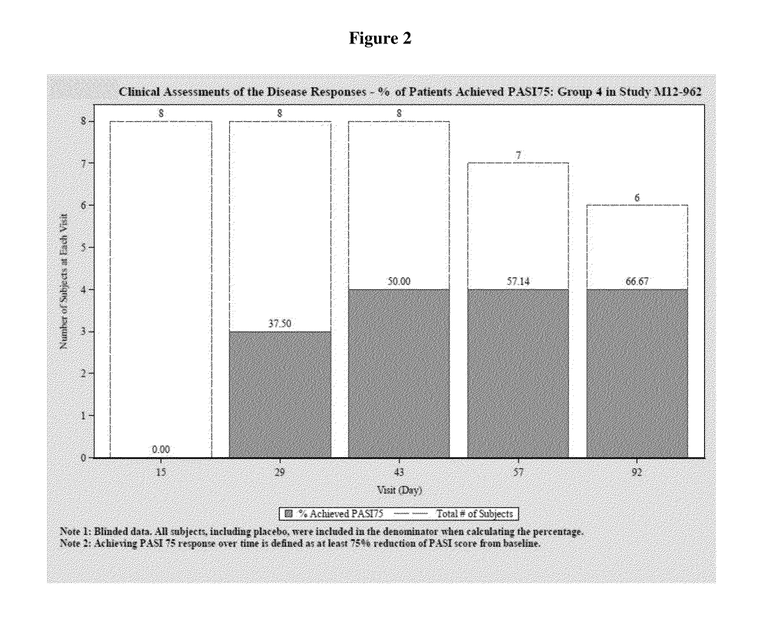

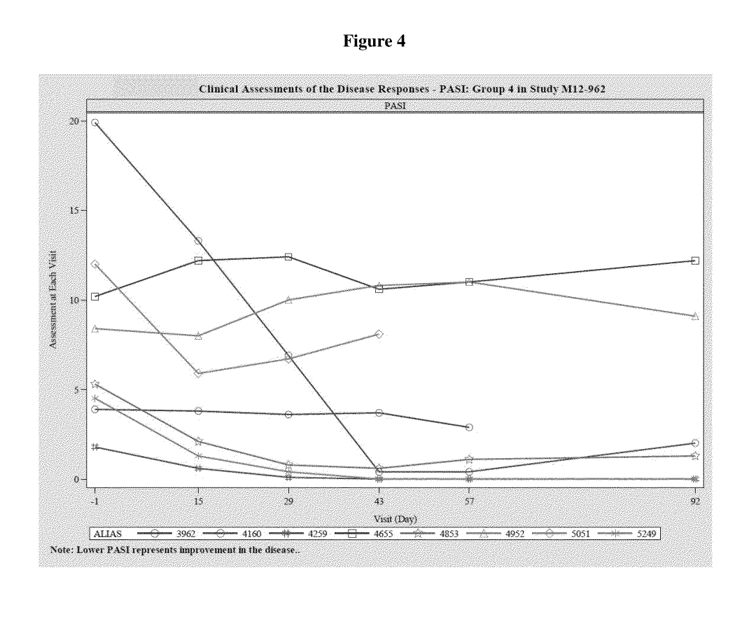

Data in FIG. 2 show that at visit days 29, 43, 57 and 92 the percent of subjects achieving PASI75 score was 37.50%, 50.00%, 57.14%, and 66.67%. Importantly, subjects showed an improvement in PASI75 score for each successive visit day during this period. Much like the data described for the Physician Global Assessment for Psoriasis, the data in FIG. 2 for each group (6-8 subjects for each visit) included two placebo subjects. Thus, there is a strong indication that if these placebo subjects had actually been administered ABT-122 DVD-Ig binding protein that the therapeutic response would have been even greater. FIG. 4 shows individual subject PASI75 score. A comparison of an individual having psoriasis and then the same individual after ABT-122 treatment shows that the binding protein markedly reduced the plaques visible on the skin.

### Example 3

### Phase 2 Study to Investigate the Safety, Tolerability and Efficacy of ABT-122 in Subjects with Active Psoriatic Arthritis Who have an Inadequate Response to Methotrexate

A Phase 1 first-in-human study in healthy volunteers with ABT-122 demonstrated good tolerability following single dose administration of up to 10 mg/kg by the IV route and up to 3 mg/kg by the SC route. Blinded safety data from studies in another disease indications show that ABT-122 was well tolerated following ABT-122 up to 3 mg/kg EW for eight doses in both studies. The safety review committees and investigators for both studies found an acceptable safety and tolerability profile for ABT-122 through 3 mg/kg EW dosing, subcutaneously, for eight doses.

The risk of a hypersensitivity reaction or other post-dose systemic reactions in this study is minimized by protocol defined inclusion and exclusion criteria, study design features, and specified safety monitoring procedures. In addition, the risk of a hypersensitivity reaction is further mitigated by the following considerations: 1) ABT-122 binds principally to soluble cytokines (TNF and IL-17); 2) ABT-122 acts as an antagonist or neutralizing antibody, and not as an agonist; 3) ABT-122 does not trigger cytokine release in vitro; and 4) preclinical hypersensitivity reactions were not observed in any animals dosed by the subcutaneous route of administration.

Based on the emerging safety data from the previous ABT-122 study, the risk for PsA patients receiving multiple doses of ABT-122 by the subcutaneous route is considered manageable and acceptable.

Both of TNF-α and IL-17 have shown to be important contributors to the disease manifestations of PsA, including skin features, the peripheral joint features, and features in the spine and enthesitis, but the treatment of PsA using a bispecific anti-TNF-α/IL-17 molecule has not been demonstrated previously.

A Phase 2 randomized, double-blind, double-dummy, active- and placebo-controlled, parallel-group multicenter study was designed to assess the safety, tolerability, efficacy, pharmacokinetics and immunogenicity of varying doses of ABT-122 given on background methotrexate (MTX). Eligible male and female subjects with PsA are selected to participate in the study according to the selection criteria. This study (M14-197) also includes exploratory biomarkers to investigate other pharmacodynamic effects of ABT-122.

This study includes a 30-day screening period conducted within 30 days of the first dose of study drug and a 12-week double-blind, active- and placebo-controlled treatment period This study is designed to enroll approximately 220 subjects to meet scientific and regulatory objectives without enrolling an undue number of subjects in alignment with ethical considerations.

### Subject Population

This study enrolls male and female subjects who had been diagnosed with PsA for at least three months and are on a stable regimen of MTX for at least four weeks.

### Inclusion Criteria:

1. Adult male or female, 18 years of age or older.
2. PsA diagnosis of at least three months duration prior to the date of first screening with Classification of Psoriatic Arthritis (CASPAR) confirmed diagnosis at Screening.
3. Have active psoriasis defined by at least one psoriasis lesion ≧2 cm diameter in areas other than the axilla or groin.
4. Have active arthritis defined by minimum disease activity criteria:
   ≧3 swollen joints (based on 66 joint counts) at Screening,
   ≧3 tender joints (based on 68 joint counts) at Screening.
5. On a stable dose of methotrexate (MTX) defined as:
   oral or parenteral treatment ≧3 months,
   on a stable dose with an unchanged mode of application for at least 4 weeks prior to baseline,
   stable MTX dose of ≧10 mg/week and ≦the upper limit of the applicable approved local label,
   subject can also be on stable doses of nonsteroidal anti-inflammatory drugs (NSAIDs), sulfasalazine and/or hydroxychloroquine as long as they are also on methotrexate.

### Exclusion Criteria:

1. Prior exposure to any tumor necrosis factor inhibitors including adalimumab.
   Up to 30% (approximately 66 subjects) with prior exposure to a TNF inhibitor may be enrolled if the TNF inhibitor was not discontinued due to lack of efficacy or safety concerns. Subjects must be washed out for at least 5 half-lives of these drugs prior to the Baseline visit.
   Subjects on prior adalimumab may not be enrolled in the study.
   Prior exposure to other non-TNF inhibitor biological disease-modifying antirheumatic drugs (DMARDs) will be permitted if the subject is washed out at least 5 half-lives of these drugs prior to the baseline visit.
2. Current treatment with traditional oral DMARDs, including conventional synthetic DMARDs (csDMARDs), (except for concomitant treatment with sulfasalazine and/or hydroxychloroquine in addition to MTX). Oral DMARDs must be washed out for at least 5 half-lives of a drug apart from MTX prior to the Baseline visit.
   Subject could have been exposed to prior Janus kinase (JAK) inhibitors so long as they have been off therapy for at least 5 half-lives.
3. Stable prescribed dose of oral prednisone or prednisone equivalent >10 mg/day within the 30 days of the Baseline visit.
4. Intra-articular or parenteral administration of corticosteroids in the preceding 4 weeks of the Baseline visit. Inhaled corticosteroids for stable medical conditions are allowed.
5. Laboratory values of the following at the Screening Visit:
   confirmed hemoglobin <9 g/dL for males and <8.5 g/dL for females,
   absolute neutrophil count (ANC)<1500 mm³, (or <1200 cells/μL for subjects of African descent who are black),
   aspartate aminotransferase (AST) or alanine aminotransferase (ALT)>1.5× the upper limit of normal (ULN) or bilirubin ≧3 mg/dL,
   serum creatinine >1.5× the ULN,
   platelets <100,000 (cells/mm3) (10⁹/L),
   clinically significant abnormal screening laboratory results as evaluated by the Investigator.

### Methodology

The ABT-122 dose is prepared in a formulation buffer suitable for manufacturing the pharmaceutical form at a concentration of 100 milligrams (mg). See Table 5. Adalimumab (Humira®), a recombinant human antibody that has a fully human heavy and light chain, is obtained as a pre-filled syringe with a solution for injection (40 mg/0.8 ml). See Table 6.

The study includes a screening period, then the 12 week double blind treatment period, and a follow-up visit period after the last treatment visit. Subjects are randomized in a 3:3:3:1 fashion to one of four dosing arms. An ABT-122 placebo dose is subcutaneously administered EW for weeks 0-12 for the blinded portion of the study (66 subjects). The three other dosing arms (two doses of ABT-122 and one dose of adalimumab) are administered by subcutaneous injection to 66 subjects in each dosing arm. The dosing arms for the study include: adalimumab (Humira®) 40 mg dose administered every other week (EOW); the ABT-122 anti-IL-17/TNF DVD-Ig protein dose A (120 mg) administered every week (EW); and the ABT-122 anti-IL-17/TNF DVD-Ig protein dose B (240 mg) administered every week (EW).

Subjects receive ABT-122 or matching placebo for ABT-122 weekly as well as either adalimumab or its matching placebo for adalimumab administered EOW through Treatment Week 11. Subjects receive no more than 3 injections per visit during the study and are required to stay at the site for at least 1 hour after dosing for safety monitoring. Subjects continue their weekly stable dose of MTX and folate.

The areas to avoid for SC injections include: any blood vessels, thickening or tenderness of skin, scars, fibrous tissue, lesions, stretch marks, bruises, redness, nevi, or other skin imperfections. Injection sites should be at least 1 inch apart and at least 2 inches from the navel.

TABLE 4
Identity of Investigational Product (ABT-122)

| Investigational | ABT-122 Powder for Solution for | Placebo for ABT-122 Lyophilized |
|---|---|---|
| Product | Injection, 100 mg, 1.0 mL Vial | Powder for Solution for Injection Vial |
| Dosage Form | Lyophilized powder for solution for | Lyophilized powder for solution for |
| | injection in vials | injection in vials |
| Formulation | ABT-122, Sucrose, Histidine, | Sucrose, Histidine, Polysorbate 80, |
| | Polysorbate 80, Water for injections, | Water for injections, Hydrochloric acid |
| | Hydrochloric acid added as necessary | added as necessary to adjust pH |
| | to adjust pH | |
| Strength (mg) | 100 mg/mL when reconstituted with | N/A when reconstituted with 1.2 mL of |
| | 1.2 mL of sterile water for injection | sterile water for injection |
| Mode of | Subcutaneous injection | Subcutaneous injection |
| Administration | | |

TABLE 5
Identity of Investigational Product - Adalimumab and Placebo

| | Adalimumab Solution for | Placebo for Adalimumab Solution |
|---|---|---|
| Investigational | Injection, 50 mg/mL (0.8 mL) Pre- | for Injection, 0.8 mL Pre-Filled |
| Product | Filled Syringe | Syringe |
| Dosage Form | Solution for injection in pre-filled | Solution for injection in pre-filled |
| | syringe | syringe |
| Formulation | Adalimumab/Mannitol, Citric acid | Mannitol, Citric acid monohydrate, |
| | monohydrate, Sodium citrate, | Sodium citrate, Disodium phosphate |
| | Disodium phosphate dihydrate, | dihydrate, Sodium dihydrogen |
| | Sodium dihydrogen phosphate | phosphate dihydrate, Sodium |
| | dihydrate, Sodium chloride, | chloride, Polysorbate 80, Water for |
| | Polysorbate 80, Water for injections, | injections, Sodium hydroxide added |
| | Sodium hydroxide added as | as necessary to adjust pH |
| | necessary to adjust pH | |
| Strength (mg) | 40 mg/0.8 mL | N/A (0.8 mL) |
| Mode of | Subcutaneous injection | Subcutaneous injection |
| Administration | | |

The mode of administration for adalimumab and placebo for adalimumab is subcutaneous injection. Adalimumab, solution for injection, 50 mg/mL (0.8 mL) and placebo for adalimumab, solution for injection, 0.8 mL do not require any reconstitution before use. The ABT-122 drug product (active or placebo) is provided as a lyophilized powder. Each vial of ABT-122 is reconstituted with 1.2 mL of sterile water for injection to provide a 100 mg/mL ABT-122 active or placebo solution. The reconstituted drug is administered via subcutaneous (SC) injection. The total volume administered is dependent on the assigned dose level.

### Blinding

The study is conducted in a double-blind manner such that the Principal Investigator and subjects are blinded to the treatment assignments. All study site personnel, except for the study drug preparation designee or pharmacist, remain blinded to the treatment.

### 12-Week Treatment Period

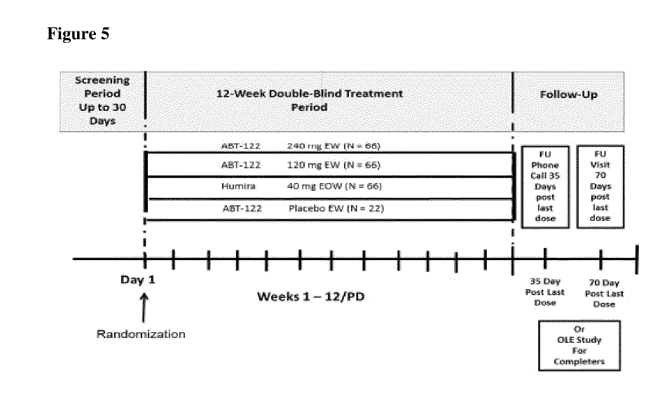

This treatment period begins at the Baseline visit (day 1) and ends at the week 12 visit (FIG. 5). At the Baseline visit, subjects who meet all the inclusion criteria and none of the exclusion criteria described herein are enrolled into the study and randomized to double-blind treatment. During this period of the study, subjects visit the study site weekly through week 12. A ±2-day window is permitted around scheduled study visits. The last dose of study drug is given during the week 11 visit. Subjects may discontinue study drug treatment at any time during study participation. Subjects who end study participation early have a Premature Discontinuation (PD) Visit and complete the procedures outlined herein as soon as possible after the last dose of study drug and preferably prior to the administration of any new therapies.

### Follow-Up Period

Subjects have a follow-up phone call approximately 35 days after the last dose of study drug and a follow-up visit approximately 70 days after the last dose of study drug with the exception of those subjects who roll over into a separate open-label extension (OLE) protocol.

### Collection of Samples:

Blood samples from all subjects enrolled are collected to measure pharmacokinetic variables. Subjects have blood drawn/collected for pharmacokinetic assessment at specified study visits. Pharmacokinetic (PK) samples are collected at specified visits through week 12 and for weeks afterward.

Blood samples are collected to assess the mechanism of action of the anti-IL-17/TNF DVD-Ig protein and a disease response. Samples are analyzed for measurement of non-genetic markers related to disease activity/prognosis of PsA, autoimmunity/inflammation, and/or response to medications, including the anti-IL-17/TNF DVD-Ig protein or drug of this class.

Patients are analyzed for safety and adverse effects throughout the study. Blood samples are collected to determine the presence of ADA and measurement of ADA titers for the assessment of immunogenicity.

DNA samples are collected from subjects who provide informed consent. These samples may be analyzed for genetic factors contributing to the subject's response to the anti-IL-17/TNF DVD-Ig protein, or other study treatment, in terms of pharmacokinetics, immunogenicity, tolerability and safety. Such genetic factors may include genes for drug metabolizing enzymes, drug transport proteins, genes within the target pathway, or other genes believed to be related to drug response. Some genes currently insufficiently characterized or unknown may be understood to be important at the time of analysis. The samples are analyzed as part of a multi-study assessment of genetic factors involved in the response to the anti-IL-17/TNF DVD-Ig protein or drugs of this class. The samples may also be used for the development of diagnostic tests related to the anti-IL-17/TNF DVD-Ig protein (or drugs of this class).

### Pharmacokinetics and Immunogenicity:

ABT-122 serum concentrations are determined during the twelve week treatment period and the follow-up visit as described herein. A mixed-effects modeling approach is used to estimate the population central values and the empirical Bayesian estimates of the individual values for ABT-122 apparent clearance (CL/F) and volume of distribution (V/F). Additional parameters may be estimated if useful in the interpretation of the data. Pharmacokinetic data from this study is combined with data from other ABT-122 studies for the population pharmacokinetic analysis. Multiple measurements of anti-drug antibodies (ADA) are collected for each subject during the 12-week double-blind randomized period. The percentage of subjects with anti-drug antibody (ADA) is calculated for each dosing regimen. As appropriate, the effect of ADA on ABT-122 pharmacokinetics and efficacy is explored. For subjects randomized to adalimumab, serum concentrations of adalimumab are determined.

### Study Drug Exposure

Exposure to study drug is summarized for all subjects who receive at least one dose of study drug. The duration (days) of study drug treatment is summarized with the mean, standard deviation, median and range for each treatment group. The duration of treatment is defined as the difference between the dates of the first and last doses of the treatment plus one day. Study drug compliance of the blinded study drug is summarized for each treatment group. Compliance is defined as the number of injections taken divided by the number of injections a subject is supposed to take during the Treatment Phase of the study. Subjects with missing data for the number of injections returned are excluded from the summary.

### Outcome Measures

The primary outcome measure is change in American College of Rheumatology Response Rate (ACR) 20 at week 12 of ABT-122 treated subjects with comparison versus placebo treated subjects. ACR criteria measure improvements in tender and swollen joint counts, from week 0 to week 12 patient assessments of pain, global disease activity and physical function, physician global assessment of disease activity and acute phase reactant. The secondary outcome measures include change in ACR 20 of ABT-122 in comparison to Adalimumab for week 0 to week 12. Additional secondary criteria that may be analysed include for example, proportion of subjects achieving ACR50 responder status at week 12; empirical cumulative distribution function of ACRn at week 12 (ACRn measures percentage improvements in tender and swollen joint at week 12 counts, patient assessments of pain, global disease activity and physical function, physician global assessment of disease activity and acute phase reactant); change in the disease activity score (DAS) 28 (determined by disease activity score using 28 joint counts (DAS28) and from week 0 to week 12 high-sensitivity C-reactive protein (hsCRP) lab test); change in psoriatic arthritis disease activity score or PASDAS (determined by tender or swollen joint counts, patient-reported From in week 0 to week 12 outcome and hsCRP lab test); change in psoriasis target lesion score (determined by erythema, plaque scaling and plaque thickness scores) from week 0 to week 12; and proportion of subjects achieving ACR70 responder status at week 12.

### Efficacy Analysis

### Primary Efficacy Variable

The primary endpoint of this study is the ACR response rates for the ACR20 at week 12. A subject is considered an ACR20 responder if:

1. The counts for both SJC (66 joints count) and TJC (68 joints count) have reduced from Baseline by 20% or more; and
2. At least three of the five remaining ACR core set measures show reduction of 20% or more in baseline assessment:
Patient's Assessment of Pain VAS,
Patient's Global Assessment of Disease Activity for arthritis (PtGA) VAS,
Physician's Global Assessment of Disease Activity for arthritis (PGA) VAS,
Patient's Assessment of Physical Function by Heath Assessment Questionnaire—(HAQ-S), and,
Acute Phase Reactant (hsCRP).

### Analysis of Primary Endpoint

The primary efficacy analysis is the comparison between ABT-122 treatment and the placebo group on ACR20 response rate at week 12. The primary efficacy analysis is conducted with the modified ITT analysis set. Subjects who discontinue treatment prior to week 12 are categorized according to LOCF. Estimates of the treatment effects in response rate and the associated 80% and 95% confidence interval for each treatment group are calculated using the Agrestil-Coull method. The comparisons between ABT-122 and control groups (adalimumab and placebo) are conducted using chi-square test or Fisher's exact test when normal approximation is not appropriate. The comparison between each ABT-122 treatment group and placebo is used to determine whether the primary objective is met. The secondary comparison on ACR20 response rate at week 12 between each ABT-122 treatment group and adalimumab helps inform likelihood that ABT-122 could be superior to adalimumab in future trials.

The following sensitivity analyses for the primary endpoint are conducted:
The primary analysis is repeated using NRI imputation method. Subjects who discontinue prior to week 12 are considered as non-responders.
The primary analysis is repeated using mixed-imputation. Subjects who discontinue prior to week 12 due to lack of efficacy or adverse events are considered non-responders. Subjects who discontinue for other reasons are categorized according to LOCF.
The primary analysis is repeated using observed cases without imputation.

### Secondary Efficacy Variables

The secondary endpoints include:
ACR50/70 response rate at week 12,
Empirical cumulative distribution function of ACRn at week 12,
Change from Baseline in DAS28 (hsCRP) at week 12,
Change from Baseline in PASDAS at week 12, and
Change from Baseline in Psoriasis Target Lesion Score at week 12.

The ACR50 and ACR70 response rates are similarly defined as ACR20, with thresholds of response set at 50% and 70%, respectively.

ACRn is defined as the average of the following three variables:
1. The percentage improvement in TJC;
2. The percentage improvement in SJC; and
3. The median percentage improvement in the following five remaining ACR core set measures:
   Patient's Assessment of Pain (VAS),
   Patient's Global Assessment of Disease Activity for Arthritis (VAS),
   Physician's Global Assessment of Disease Activity for Arthritis (VAS),
   Patient's Assessment of Physical Function by Health Assessment Questionnaire—(HAQ-S), and
   hsCRP

Empirical cumulative distribution function (ECDF) for ACRn are calculated and plotted for each treatment group and compared by using Kolmogorov-Smirnov (KS) test. ECDF for each treatment group is defined as the number of subjects with ACR response ≦t divided by the number of the subjects in the treatment group for all t from 0 to 1.

DAS28 (hsCRP) score is determined based on a continuous scale of combined measures of TJC, SJC, Patient Global Assessment of Disease Activity for arthritis (PtGA) (in mm), and hsCRP (in mg/L) at week 12.

DAS28(hsCRP)=0.56√(“TJC28”)+0.28√(“SJC28”)+0.36 ln(hsCRP+1)+0.014PtGA+0.96,

where √ is square root and ln is natural log.

Psoriatic Disease Activity Score (PASDAS) is a continuous scale of combined joint assessment, PRO and hsCRP measurements at week 12.

PASDAS=(((0.18√(“PGA”))+0.159√(“PtGA”)−0.253√(“SF36-PCS”)+0.101 ln (SJC+1)+0.048 ln(TJC+1)+0.23 ln(Leeds Enthesitis Count+1)+0.37 ln(tender dactylitis count+1)+0.102 ln(hsCRP+1)+2)*1.5,

where √ is square root and ln is natural log. PGA is physician global assessment of disease activity for arthritis and PtGA is patient global assessment of disease activity for arthritis. SF36-PCS is the physical component scale in SF36 instrument.

Target lesion score for psoriasis in patients with psoriatic arthritis is calculated by adding the scores in the instrument.

### Analysis of Secondary Endpoints

For binary endpoints including the proportion of subjects achieving ACR50/70 at week 12, frequencies and relative frequencies in each group are reported. A chi-square test or Fisher's exact test (when normal approximation is not appropriate) for comparisons of proportions between each of the ABT-122 treatment groups and the control groups is performed at week 12. For continuous endpoints including DAS28 (hsCRP), PASDAS, Target Lesion Score, the mean, standard deviation, median, and range are reported at week 12. Overall and pairwise comparisons for each of the ABT-122 treatment groups and the control groups are carried out using the analysis of covariance (ANCOVA) models with treatment group as a factor and the corresponding baseline score as a covariate at week 12, respectively.

The ACRn is calculated and compared between each of the ABT-122 treatment groups and the control groups at week 12. Empirical cumulative distribution functions for ACRn for ABT-122 and control groups are plotted and compared using a Kolmogorov-Smirnov (KS) test.

### Exploratory Endpoints

Additional efficacy endpoints including Minimal Disease Activity (MDA) PASI responses in subjects with PASI >3 at baseline, dactylitis (dactylitis score), enthesitis (SPARCC), BASDAI (stratified by the presence/absence of inflammatory back pain) by treatment groups at week 12. Additional exploratory endpoints include for example ACR20/50/70 response rates by treatment groups across 12 weeks by study visits; change from baseline for DAS28 (hsCRP), PASDAS, Psoriasis Target Lesion Scores, PASI, dactylitis and enthesitis (SPARCC) by treatment groups across 12 weeks by study visits; change from baseline for the individual components of each of the composite responses measures (component measures of ACR responses) by treatment groups across 12 weeks by study visits; change from baseline in measures of quality of life, function and work (SF-36v2, HAQ-S, BASDAI, Fatigue Numeric Rating Scale, Sleep Quality Scale and Self-Assessment of Psoriasis Symptoms [SAPS]) by study visits; and change from baseline in biomarkers.

### Exploratory Endpoints

Additional efficacy endpoints including Minimal Disease Activity (MDA) PASI responses in subjects with PASI >3 at Baseline, dactylitis (dactylitis score), enthesitis (SPARCC), BASDAI (stratified by the presence/absence of inflammatory back pain) by treatment groups at week 12.
ACR20/50/70 response rates by treatment groups across 12 weeks by study visits.
Change from baseline for DAS28 (hsCRP), PASDAS, Psoriasis Target Lesion Scores, PASI, dactylitis and enthesitis (SPARCC) by treatment groups across 12 weeks by study visits.
Change from baseline for the individual components of each of the composite responses measures (component measures of ACR responses by treatment groups across 12 weeks by study visits.
Change from baseline in measures of quality of life, function and work (SF-36v2, HAQ-S, BASDAI, Fatigue Numeric Rating Scale, Sleep Quality Scale and SAPS) by study visits.
Change from baseline in biomarkers.

### Analysis of Exploratory Endpoints

For binary endpoints, frequencies and relative frequencies in each group are reported. A chi-square test or Fisher's exact test (when normal approximation is not appropriate) for comparisons of proportions between each of the ABT-122 treatment groups and the control groups is performed at week 12.

For continuous endpoints, the mean, standard deviation, median, and range are reported at week 12. Overall and pairwise comparisons for each of the ABT-122 treatment groups and the control groups is carried out using the analysis of covariance (ANCOVA) models with treatment group as a factor and the corresponding baseline score as a covariate at week 12, respectively.

### Statistical Methods:

### Efficacy

With a 3:1 randomization ratio, the sample size in ABT-122 treatment group and placebo provides more than 80% power to detect a 50% increase of the ACR20 response rate compared to placebo with two-sided 5% alpha, assuming ACR20 response rate on placebo is 20%. Approximately 66 subjects in each of the ABT-122 dose arms and adalimumab arm would provide 80% power with one-sided 10% alpha to detect a 20% increase of the ACR20 and ACR50 response rates compared to adalimumab, assuming ACR20/50 response rates for adalimumab are 53.5% and 45%, respectively. Empirical cumulative distribution function of ACRn at week 12 for each treatment group is calculated and compared.

For ACR responder analysis, response rate and 80% and 95% confidence interval associated with it are summarized using Agristi-Coull method. The comparisons between ABT-122 and control groups (adalimumab and placebo) is conducted using chi-square test or Fisher's exact test when normal approximation is not appropriate. Unless otherwise specified, statistical tests are conducted with one-sided significance level of 0.1 for efficacy analyses involving comparison versus adalimumab and one-sided significance level of 0.025 for efficacy analyses involving placebo. A test is deemed nominal significant if the P value rounded to two decimal places is less than or equal to 0.1 or 0.025 unless otherwise specified. The SAS System is used to perform the statistical analyses. No multiplicity adjustment is performed. Empirical cumulative distribution functions for ACRn for ABT-122 and adalimumab are plotted and compared using Kolmogorov-Smirnov (KS) test.

### Pharmacokinetic Variables

Serum ABT-122 or adalimumab concentrations are determined weekly during the 12-week treatment period and at the follow-up visit as outlined in Table 6. A mixed-effects modeling approach is used to estimate the population central values and the empirical Bayesian estimates of the individual values for ABT-122 apparent clearance (CL/F) and volume of distribution (V/F). Additional parameters are estimated if useful in the interpretation of the data. Data from this study may be combined with data from other ABT-122 studies for the population analysis.

### Pharmacokinetic and Exposure-Response Analyses

Individual ABT-122 and adalimumab serum concentrations at each study visit where PK samples are collected are tabulated and summarized with appropriate statistical methods. In addition, ADA titers are tabulated for each subject at the respective study visits. Data from this study may be combined with data from other studies for the population pharmacokinetic and exposure-response analyses. Population pharmacokinetic and exposure-response analyses of only data from this study may not be conducted. The following general methodology is used for the population pharmacokinetic analysis. Population pharmacokinetic analyses of ABT-122 are performed using the actual sampling time relative to dosing. Pharmacokinetic models are built using a non-linear mixed-effects modeling approach with NONMEM software (Version 7.2, or a higher version). The structure of the starting pharmacokinetic model is based on the pharmacokinetic analysis data from previous studies. Apparent CL/F and apparent V/F of ABT-122 are the pharmacokinetic parameters of major interest in the NONMEM analyses. If necessary, other parameters, including the parameters describing absorption characteristics, are estimated if useful in the analysis.

The evaluation criteria described below are used to examine the performance of different models:
1. The objective function of the best model is significantly smaller than the alternative model(s).
2. The observed and predicted concentrations from the preferred model are more randomly distributed across the line of unity (a straight line with zero intercept and a slope of one) than the alternative model(s).
3. Visual inspection of model fits standard errors of model parameters and change in inter-subject and intra-subject error.

Once an appropriate base pharmacokinetic model (including inter- and intra-subject error structure) is developed, empirical Bayesian estimates of individual model parameters are calculated by the posterior conditional estimation technique using NONMEM. The relationship between these conditional estimates CL/F and V/F values with only potentially physiologically relevant or clinically meaningful covariates (such as ADA class, subject age, sex, body weight, concomitant medications, possibly baseline inflammatory and disease markers) are explored using stepwise forward selection method, or another suitable regression/smoothing method at a significance level of 0.05.

After identification of all relevant covariates, a stepwise backward elimination of covariates from the full model is employed to evaluate the significance (at P<0.005, corresponding to a decrease in objective function >7.88 for one degree of freedom) of each covariate in the full model.

In general, all continuous covariates are entered in the model, initially in a linear fashion, with continuous covariates centered on the median value. Linear or non-linear relationships of primary pharmacokinetic parameters with various covariates are also explored. Relationships between exposure and clinical observations (primary or secondary efficacy or safety variables of interest) may be explored.

Additional analyses are performed if useful and appropriate. The pharmacokinetic and exposure-response analyses are presented in a separate report prior to regulatory filings for approval of ABT-122. The analyses are presented either using the data from the current study only or as part of a combined analysis of data from multiple studies of ABT-122.

### Biomarkers:

Pharmacodynamic and mRNA Biomarkers

Blood samples are collected at time points specified in Table 6 to assess the mechanism of action of ABT-122. Results from these exploratory studies are not necessarily a part of the study report.

### Disease Response Biomarkers

Subjects have additional blood and urine samples collected at time points specified in Table 6 to assess disease response. Samples are analyzed for measurement of non-genetic markers related to disease activity/prognosis of PsA, autoimmunity/inflammation, and/or response to anti-PsA medications, including ABT-122 or drug of this class.

### Skin Biopsy Biomarkers

Skin samples are collected at time points specified in Table 6 to assess biomarkers and gene expression related to disease activity/prognosis of PsA, autoimmunity/inflammation, and/or response to anti-PsA medications, including ABT-122 or drug of this class.

### Clinical Assessments

To explore the potential disease response signals, the following clinical assessments are obtained: swollen joint count, tender joint count, physician's global assessment of disease activity for arthritis VAS, patient's global assessment of disease activity for arthritis VAS, patient's assessment of pain VAS, physician's global assessment for psoriasis VAS, PASI score, target lesion score, dactylitis/enthesites assessments, as well as the following patient reported outcomes questionnaires: HAQ-S, SF-36v2, BASDAI, Fatigue NRS, Sleep Quality Scale and SAPS.

### Safety

Safety evaluations include adverse event monitoring, physical examinations, vital sign measurements, electrocardiogram, and clinical laboratory testing (hematology, chemistry, and urinalysis) as a measure of safety and tolerability. Toxicity management guidelines are provided within the protocol.

### Anti-Drug Antibody Assessment

ABT-122 is a monoclonal antibody-like molecule and such molecules can lead to immunogenic responses in patient populations. The most common manifestation of this is the development of anti-drug antibodies (ADA). When these ADA have clinical effects, the most common manifestation is increased drug clearance. Occasionally ADA can lead to interference with drug action (neutralization). Both the increased drug clearance and interference with action lead to reduced clinical effectiveness. To date these effects for ABT-122 have been rare occurrences. The collection of PK and ADA is part of this protocol. Another described immunological effect of monoclonal antibodies is the development of injection site reactions (ISRs). While the current multiple ascending dose studies remain blinded, ISRs have been seen rarely in the clinical studies to date.

### Pharmacogenetic Variables:

DNA samples may be sequenced and data analyzed for exploratory genetic factors contributing to the disease or the subject's response to ABT-122, or other study treatment, in terms of pharmacokinetics, efficacy, tolerability and safety. Such genetic factors may include genes for drug metabolizing enzymes, drug transport proteins, genes within the target pathway, or other genes believed to be related to disease or drug response. Some genes currently insufficiently characterized or unknown may be understood to be important at the time of analysis. The samples may be analyzed as part of a multi-study assessment of genetic factors involved in the response to ABT-122 or drugs of this class. The samples may also be used for the development of diagnostic tests related to disease or ABT-122 (or drugs of this class).

Removal of Subjects from Therapy or Assessment

### Discontinuation of Individual Subjects

A subject may withdraw from the study at any time. The Investigator may discontinue any subject's participation for any reason, including an adverse event, safety concerns or failure to comply with the protocol.

Subjects are withdrawn from the study if any of the following occur:
Clinically significant confirmed abnormal laboratory results or adverse events, which rule out continuation of the study medication, as determined by the Investigator and the SDP
A subject experiences a serious adverse event for which there is no clear alternative explanation (e.g., the subject is a victim of a motor vehicle accident).
A subject experiences a moderate/grade 2 adverse event of vasculitis for which there is no clear alternative explanation.
Subject experiences grade 3 or greater, severe, or life threatening injection site reaction (ISR) (as defined by the Rheumatology Common Toxicity which includes prolonged induration, superficial ulceration and includes thrombosis or major ulceration or necrosis requiring surgery.
The Investigator believes it is in the best interest of the subject.
The subject requests withdrawal from the study.
Inclusion and exclusion criteria violation was noted after the subject started study drug, when continuation of the study drug would place the subject at risk as determined by the SDP.
Introduction of prohibited medications or dosages when continuation of the study drug would place the subject at risk as determined by the SDP.
The subject becomes pregnant while on study medication.
Subject has known dysplasia of the gastrointestinal tract (a colonoscopy is not required to enter the study) or malignancy, except for localized non-melanoma skin cancer. Discontinuation for carcinoma in-situ of the cervix is at the discretion of the Investigator.
Subject is diagnosed with lupus like syndrome, multiple sclerosis or demyelinating disease (including myelitis).
Subject is significantly non-compliant with study procedures which would put the subject at risk for continued participation in the trial in consultation with the SDP.
A subject has a confirmed platelet count <50,000 cells/mm³.

### Adverse Events

An adverse event (AE) is defined as any untoward medical occurrence in a patient or clinical investigation subject administered a pharmaceutical product and which does not necessarily have a causal relationship with this treatment. An adverse event can therefore be any unfavorable and unintended sign (including an abnormal laboratory finding), symptom, or disease temporally associated with the use of a medicinal (investigational) product, whether or not the event is considered causally related to the use of the product.

The investigator monitors each subject for clinical and laboratory evidence of adverse events on a routine basis throughout the study. The investigator assesses and records any adverse event in detail including the date of onset, event diagnosis (if known) or sign/symptom, severity, time course (end date, ongoing, intermittent), relationship of the adverse event to study drug, and any action(s) taken. For serious adverse events considered as having “no reasonable possibility” of being associated with study drug, the investigator provides another cause of the event. For adverse events to be considered intermittent, the events must be of similar nature and severity. Adverse events, whether in response to a query, observed by site personnel, or reported spontaneously by the subject are recorded.

The following AE eCRFs are collected in this study:
Hypersensitivity Reaction AE page (with supplemental Anaphylaxis page)
Hepatic-related AE page
Cardiovascular AE page

### Serious Adverse Events

If an adverse event meets any of the following criteria, it is reported as a serious adverse event (SAE) within 24 hours of the site being made aware of the serious adverse event.

Death of Subject—An event that results in the death of a subject.
Life Threatening—An event that, in the opinion of the investigator, would have resulted in immediate fatality if medical intervention had not been taken. This does not include an event that would have been fatal if it had occurred in a more severe form.
Hospitalization or Prolongation of Hospitalization—An event that results in an admission to the hospital for any length of time or prolongs the subject's hospital stay. This does not include an emergency room visit or admission to an outpatient facility.
Congenital Anomaly—An anomaly detected at or after birth, or any anomaly that results in fetal loss.
Persistent or Significant Disability/Incapacity—An event that results in a condition that substantially interferes with the activities of daily living of a study subject. Disability is not intended to include experiences of relatively minor medical significance such as headache, nausea, vomiting, diarrhea, influenza, and accidental trauma (e.g., sprained ankle).
Important Medical Event Requiring Medical or Surgical Intervention to Prevent Serious Outcome—An important medical event that may not be immediately life-threatening or result in death or hospitalization, but based on medical judgment may jeopardize the subject and may require medical or surgical intervention to prevent any of the outcomes listed above (i.e., death of subject, life-threatening, hospitalization, prolongation of hospitalization, congenital anomaly, or persistent or significant disability/incapacity). Additionally, any elective or spontaneous abortion or stillbirth is considered an important medical event. Examples of such events include allergic bronchospasm requiring intensive treatment in an emergency room or at home, blood dyscrasias or convulsions that do not result in inpatient hospitalization, or the development of drug dependency or drug abuse.

### Relationship to Study Drug

The Investigator uses the following definitions to assess the relationship of the adverse event to the use of study drug:
Reasonable Possibility—An adverse event where there is evidence to suggest a causal relationship between the study drug and the adverse event.
No Reasonable Possibility—An adverse event where there is no evidence to suggest a causal relationship between the study drug and the adverse event.

### Adverse Event Collection Period

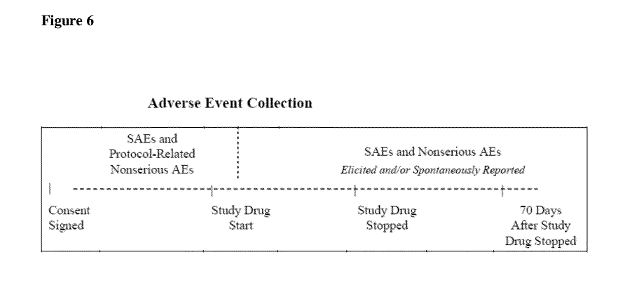

All adverse events identified at the 70-day follow-up visit are collected as source data to be evaluated and reported (FIG. 6). Thus, all SAEs and non-serious AEs, reported during the 70-day follow-up visit must be captured in the clinical database. The end of trial is the last subject contact, i.e., the 70-day follow-up visit. Adverse event information is collected as shown below.

TABLE 6
Study Activities

| | | | | | | | | | | | | | | | Follow-Up | |
|---|---|---|---|---|---|---|---|---|---|---|---|---|---|---|---|---|
| | | | | | | | | | | | | | | | Period^{a} | |
| | | | | | | | | | | | | | | | 35-Day | |
| | Screening | | | | | | | | | | | | | | Follow- | 70-Day |
| | Within | Treatment Period | | | | | | | | | | | | | Up | Follow- |
| | 30 Days | | | | | | | | | | | Wk | Wk | Wk | Phone | Up |
| | Prior to | Wk 0 | Wk 1 | Wk 2 | Wk 3 | Wk 4 | Wk 5 | Wk 6 | Wk 7 | Wk 8 | Wk 9 | 10 | 11 | 12/PD | Call | Visit |
| Procedure | 1^{st} Dose | D1 | D 8 | D 15 | D 22 | D 29 | D 36 | D 43 | D 50 | D 57 | D 64 | D 71 | D 78 | D 85 | D 113 | D 148 |
| Informed Consent^{b} | X | | | | | | | | | | | | | | | |
| Medical/Surgical | X | X^{c} | | | | | | | | | | | | | | |
| History | | | | | | | | | | | | | | | | |
| Physical Exam^{d} | X | | | | | | | | | | | | | X | | X |
| Vital Signs^{e} | X | X | X | X | X | X | X | X | X | X | X | X | X | X | | X |
| 12-Lead ECG | X | X | | | | | | | | | | | | X | | |
| Chest X-Ray^{f} | X | | | | | | | | | | | | | | | |
| Latent TB Risk | X | | | | | | | | | | | | | | | |
| Factor Questionnaire | | | | | | | | | | | | | | | | |
| PPD Skin Test or | X | | | | | | | | | | | | | | | |
| QuantiFERON-TB | | | | | | | | | | | | | | | | |
| Gold Test | | | | | | | | | | | | | | | | |
| Blood Chemistry | X | X^{g} | | | | X^{g} | | | | X^{g} | | | | X | | X |
| Hematology (CBC) | X | X^{g} | | X^{g} | | X^{g} | | X^{g} | | X^{g} | | X^{g} | | X | | X |
| Urinalysis | X | X^{g} | | | | X^{g} | | | | X^{g} | | | | X | | X |
| hsCRP for DAS | X | X | | X | | X | | | | X | | | | X | | X |
| HIV^{h} | X | | | | | | | | | | | | | | | |
| Hepatitis Panel | X | | | | | | | | | | | | | | | |
| (HBsAg, HBsAb, | | | | | | | | | | | | | | | | |
| HBcAb, HCV, | | | | | | | | | | | | | | | | |
| HBV) | | | | | | | | | | | | | | | | |
| Pregnancy Test | X (s) | X | | | | | | | | | | | | X | | X (u) |
| U = Urine | | (u, s)ⁱ | | | | | | | | | | | | (u, s)ⁱ | | |
| S = Serum | | | | | | | | | | | | | | | | |
| Inflammatory Back | X | | | | | | | | | | | | | | | |
| Pain History | | | | | | | | | | | | | | | | |
| Blood Sample for | | X^{g} | | X^{g} | | X^{g} | | X^{g} | | X^{g} | | X^{g} | | X | | X |
| PK Assay^{j} | | | | | | | | | | | | | | | | |
| Blood Sample for | | X^{g} | | X^{g} | | X^{g} | | | | X^{g} | | | | X | | |
| ADA Assay^{j} | | | | | | | | | | | | | | | | |
| Disease Response | | X^{g} | | X^{g} | | X^{g} | | | | X^{g} | | | | X | | |
| (DR) Serum | | | | | | | | | | | | | | | | |
| Biomarkers | | | | | | | | | | | | | | | | |
| DR Urine | | X^{g} | | X^{g} | | X^{g} | | | | X^{g} | | | | X | | |
| Biomarkers | | | | | | | | | | | | | | | | |
| Pharmacodynamic | | X^{g} | | X^{g} | | X^{g} | | | | X^{g} | | | | X | | |
| (PD) Serum, Plasma, | | | | | | | | | | | | | | | | |
| PBMC, mRNA | | | | | | | | | | | | | | | | |
| Biomarkers | | | | | | | | | | | | | | | | |
| PD Biomarker Whole | | X^{g} | | | | X^{g} | | | | | | | | X | | |
| Blood | | | | | | | | | | | | | | | | |
| Future Research | | X^{g} | | X^{g} | | | | | | X^{g} | | | | X | | |
| Samples^{k} | | | | | | | | | | | | | | | | |
| Pharmacogenomic | | X | | | | | | | | | | | | | | |
| Blood Sample^{k} | | | | | | | | | | | | | | | | |
| Complements (C3, | | | | | | | | | | | | | | | | |
| C3a, C4, CH50), | | | | | | | | | | | | | | | | |
| cytokines (TNF, | | | | | | | | | | | | | | | | |
| IL-1β, IL-2, IL-6), | | | | | | | | | | | | | | | | |
| tryptase and hsCRP^{l} | | | | | | | | | | | | | | | | |
| 24-hour | | | | | | | | | | | | | | | | |
| methylhistamine^{j} | | | | | | | | | | | | | | | | |
| Urine Protein/ | | | | | | | | | | | | | | | | |
| Creatinine | | | | | | | | | | | | | | | | |
| Ratio^{j} | | | | | | | | | | | | | | | | |
| Tender Joint Count | X | X | | X | | X | | | | X | | | | X | | X |
| (TJC)^{m} | | | | | | | | | | | | | | | | |
| Swollen Joint Count | X | X | | X | | X | | | | X | | | | X | | X |
| (SJC)^{m} | | | | | | | | | | | | | | | | |
| Target Lesion | X | X | | | | X | | | | X | | | | X | | X |
| Score^{m} | | | | | | | | | | | | | | | | |
| PASI^{m} | X | X | | | | X | | | | X | | | | X | | X |
| Physician's Global | X | X | | | | X | | | | X | | | | X | | X |
| Assessment for Ps^{m} | | | | | | | | | | | | | | | | |
| Physician's Global | X | X | | X | | X | | | | X | | | | X | | X |
| Disease Activity for | | | | | | | | | | | | | | | | |
| Arthritis VAS | | | | | | | | | | | | | | | | |
| (PGA)^{m} | | | | | | | | | | | | | | | | |
| Patient Global | X | X | | X | | X | | | | X | | | | X | | X |
| Disease Activity for | | | | | | | | | | | | | | | | |
| Arthritis VAS | | | | | | | | | | | | | | | | |
| (PtGA)^{m} | | | | | | | | | | | | | | | | |
| Patient's Disease | X | X | | X | | X | | | | X | | | | X | | X |
| Pain VAS^{m} | | | | | | | | | | | | | | | | |
| BASDAI^{m} | | X | | | | X | | | | X | | | | X | | |
| Dactylitis^{m} | | X | | | | X | | | | X | | | | X | | |
| SPARCC/ | | X | | | | X | | | | X | | | | X | | |
| -Enthesitis^{m} | | | | | | | | | | | | | | | | |
| HAQ-S^{m} | X | X | | X | | X | | | | X | | | | X | | X |
| SF-36v2^{m} | | X | | | | X | | | | X | | | | X | | |
| Fatigue Scale^{m} | | X | | | | X | | | | X | | | | X | | |
| Sleep Quality | | X | | | | X | | | | X | | | | X | | |
| Scale^{m} | | | | | | | | | | | | | | | | |
| SAPS^{m} | | X | | | | X | | | | X | | | | X | | |
| Skin | | X | | | | | | | | | | | | X | | |
| Biopsy/Biomarker^{k} | | | | | | | | | | | | | | | | |
| Adverse Event | | X | X | X | X | X | X | X | X | X | X | X | X | X | X | X |
| Assessment | | | | | | | | | | | | | | | | |
| Study Drug Adminⁿ | | X | X | X | X | X | X | X | X | X | X | X | X | | | |

^{a}Upon regulatory and IRB approval, subjects who roll over into a separate open-label protocol will not require the 35-day follow-up phone call or the 70-day follow-up visit as part of this trial.
^{b}Perform within 30 days prior to study drug administration.
^{c}Update history.
^{d}A symptom directed physical exam should be performed when necessary and if needed for physician assessments/questionnaires.
^{e}Height and weight will be measured at the Screening visit only (with shoes off).
^{f}Chest x-ray not required at Screening if subject had a previous normal chest x-ray within 90 days of Screening or not required per local guidelines.
^{g}To be collected before dose.
^{h}If required by country regulatory authorities to confirm eligibility, subjects will be tested for HIV and documented that the test has been performed. This testing is to be done at a local laboratory. A subject will not be eligible for study participation if test results indicate a positive HIV infection. The company performing the study will not receive results from the testing and not be made aware of any positive result.
ⁱAll females of childbearing potential will have a urine sample collected at Baseline prior to study enrollment and at study discontinuation/completion. The urine samples will be tested at the site. Monthly pregnancy tests will be performed throughout the study if required by country regulatory authorities. Any subject with a positive urine pregnancy test must have a negative serum test performed at the central laboratory prior to enrollment or continuation in the study.
^{j}In the event of a suspected hypersensitivity reaction or other systemic post-dose reaction, a PK/ADA and urine samples will be collected once within 24 hours of the reaction.
^{k}Optional samples: Subject will sign additional consent forms; if the additional consent forms are not signed, no optional samples will be collected.
^{l}In the event of a suspected hypersensitivity reaction or other systemic post-dose reaction, these blood samples may be collected within 1, 3, and 24 hours of the onset of the reaction.
^{m}Prior to other procedures.
ⁿSubjects will be required to stay at the site for at least 1 hour after dosing for safety monitoring. For subjects who prematurely discontinue, study drug will not be given at the premature discontinuation visit.

### Example 4

### A Phase 2, Multicenter, Open-Label Extension (OLE) Study with ABT-122 in Active Psoriatic Arthritis Subjects Who have Completed a Preceding Study M14-197 Phase 2 Randomized Controlled Trial (RCT)

A Phase 2, multicenter, open-label extension (OLE) study is performed over 24 weeks using ABT-122 DVD-Ig binding protein. The ABT-122 is prepared using a lyophilisate in solution for injection. The drug is an antibody-like molecule in a formulation buffer suitable for manufacturing the pharmaceutical form (concentration of 100 mg/ml). Subjects are subcutaneously injected with ABT-122 (240 mg) every other week. The primary objective of the study is to assess the long term efficacy, and safety and tolerability of ABT-122 in PsA subjects on background MTX who completed the previously described Study M14-197 Phase 2 RCT. The secondary objectives are to explore the effect of continued dosing on anti-drug antibody (ADA) profiles for ABT-122 and to explore the longer term effects of ABT-122 on function, quality of life and fatigue.

### Inclusion Criteria:

1. Subjects who have completed the preceding Study M14-197 ABT-122 RCT study and have not developed any discontinuation criteria, defined herein. Subjects will be a minimum of 18 years old and a maximum of 99 years old.
2. If female, subject must have met one of the following criteria:
Postmenopausal (defined as no menses for at least 1 year).
Surgically sterile (bilateral oophorectomy or hysterectomy).
Total abstinence from sexual intercourse as the preferred lifestyle of the subject. Periodic abstinence is not acceptable.
Practicing appropriate birth control, from the time of enrollment in this study until at least 150 days after the last dose of study drug defined as at least TWO of the following methods of birth control:
   tubal ligation,
   partner vasectomy (at least 6 months earlier) (the vasectomized male partner should be the sole partner for that female subject),
   intrauterine device (IUD),
   diaphragm, contraceptive sponge or cervical cap with spermicidal jelly or cream,
   hormonal contraceptives (note, low-dose progestin-only oral contraceptives such as norethindrone 0.35 mg and lynestenol 0.5 mg are not considered adequate),
   Combined (estrogen and progestogen containing) hormonal contraception associated with inhibition of ovulation started at least 2 months prior to the first dose of study drug: oral, intravaginal or transdermal or
   double-barrier contraception* defined as: A male condom PLUS diaphragm or cervical cap used with spermicidal jelly or cream. * Note: A female condom and a male condom should not be used together. Also, because the contraceptive sponge has a high failure rate, particularly in multiparous females, it should not be considered an acceptable alternative.
      3. Male who agreed to follow one of the protocol-specified pregnancy avoidance measures below, including refraining from donating sperm, for up to 150 days post last dose of study drug:
Subject using condom and female partner(s) using an intrauterine device (IUD).
Subject using condom and female partner(s) using hormonal contraceptives (oral, vaginal, parenteral or transdermal); (note, low dose progestin-only oral contraceptives such as norethindrone 0.35 mg and lynestenol 0.5 mg are not considered adequate).
Subject using condom and female partner(s) using double-barrier method (contraceptive sponge; diaphragm or vaginal ring with spermicidal jellies, creams, or spermicide).
Total abstinence from sexual intercourse as the preferred lifestyle of the subject; periodic abstinence is not acceptable.
   4. Subjects must have voluntarily signed and dated an informed consent, approved by an Independent Ethics Committee (IEC)/Institutional Review Board (IRB), prior to the initiation of any study-specific procedures.
   5. Subject was judged to be in good health as determined by the Investigator based on the results of medical history, physical examination and laboratory profile performed.

### Exclusion Criteria:

1. Pregnant or breastfeeding or plans to become pregnant during study participation.
2. Ongoing infections at Day 1 (Week 0) that have NOT been successfully treated within 14 days.
3. Anticipated requirement or receipt of any live vaccine during study participation including up to 120 days after the last dose of study drug.
4. Current enrollment in another investigational study; with the exception of Study M14-197, which is required.
5. Consideration by the Investigator, for any reason, that the subject is an unsuitable candidate to continue to receive ABT-122.

### Exploratory Endpoints Will be Determined (Week 0 to Week 24)

American College of Rheumatology (ACR) 20 response rate by visit. The ACR20 criteria analysis may involve determining improvements in tender and swollen joint counts, patient assessments of pain, global disease activity and physical function, physician global assessment of disease activity and acute phase reactant.
ACR50 response rate by visit. ACR50 criteria analysis may involve measuring improvements in tender and swollen joint counts, patient assessments of pain, global disease activity and physical function, physician global assessment of disease activity and acute phase reactant.
ACR70 response rate by visit. ACR70 criteria analysis may involve measuring improvements in tender and swollen joint counts, patient assessments of pain, global disease activity and physical function, physician global assessment of disease activity and acute phase reactant.
Change in ACR individual component by visit.
Change in Disease Activity Score DAS28 [hsCRP] by visit. This analysis may involve determining disease activity score using 28 joint counts (DAS28) and high-sensitivity C-reactive protein (hsCRP) lab test(s).
Change in Psoriatic Disease Activity Score (PASDAS) by visit. The change in PASDAS may be determined by analysis of tender or swollen joint counts, patient reported outcome and hsCRP lab test(s).
Change in Psoriasis Area and Severity Index (PASI) by visit. This change may be determined by analyzing scores for the amount and severity of a patient's psoriasis.
Change in Psoriasis Target Lesion Score by visit. This change may be determined by analysing plaque erythema, plaque scaling and plaque thickness scores.
Change in Dactylitis Assessment by visit. This change may be analyzed by determining presence of dactylitis, swelling, and tenderness in each digit of both hands and both feet.
Change in Entheses Sites Comprising the Total Spondyloarthritis Research Consortium of Canada (SPARCC) Enthesitis Index by visit. This change may be analyzed by determining the presence and severity of enthesitis.
Change in Self-Assessment of Psoriasis Symptoms (SAPS) by visit. This change in SAPS may be determined by analysing scores given by patients regarding the severity of their psoriatic symptoms.
Change in skin biopsy/biomarkers. These skin biopsy/biomarker changes may be determined by analysing optional samples to assess changes related to disease activity/prognosis of psoriatic arthritis (PsA), autoimmunity/inflammation, and/or response to anti-PsA medications.
Change in the quality of life, function and work as measured by the SF36v2 by visit. Quality of life may be self-reported measures used to assess the physical function of the patient and how their activities are impacted by their disease.
The change in the quality of life, function and work may be measured by Bath AS Disease Activity Index (BASDAI) by visit. Quality of life may be self-reported measures used to assess the physical function of the patient and how their activities are impacted by their disease.
Change in the quality of life, function and work as measured by the Fatigue Numeric Rating Scale by visit. Quality of life may be self-reported measures used to assess the physical function of the patient and how their activities are impacted by their disease.
Change in the quality of life, function and work as measured by the Sleep Quality Scale by visit. Quality of life may be self-reported measures used to assess the physical function of the patient and how their activities are impacted by their disease.

Subjects who complete this study M14-198 or prematurely discontinue from the study are treated in accordance with the investigator's best clinical judgment. At the subject's last visit, the investigator discusses the appropriate subsequent treatment with the subject.

Samples (e.g., serum samples) are collected from the subjects and are analysed (e.g., pharmacokinetics, concentration, and immunogenicity) using methods described herein.

### INCORPORATION BY REFERENCE

The present invention incorporates by reference in their entirety techniques well known in the field of molecular biology, drug delivery, immunology, molecular biology and cell biology. These techniques include, but are not limited to, techniques described in the following publications: Ausubel et al. (eds.) (1993) Current Protocols in Molecular Biology, John Wiley & Sons, NY; Ausubel et al. (eds.) (1999) Short Protocols In Molecular Biology John Wiley & Sons, NY (ISBN 0-471-32938-X); Smolen and Ball (eds.) (1984) Controlled Drug Bioavailability Drug Product Design and Performance, Wiley, N.Y.; Giege and Ducruix (1999) Crystallization of Nucleic Acids and Proteins, a Practical Approach, 2nd ed., pp. 20 1-16, Oxford University Press, NY; Goodson (1984) Medical Applications of Controlled Release, vol. 2, pp. 115-138; Hammerling et al. (1981) Monoclonal Antibodies and T-Cell Hybridomas 563-681 (Elsevier, N.Y.; Harlow et al. (1988) Antibodies: A Laboratory Manual, (Cold Spring Harbor Laboratory Press, 2nd ed.; Kabat et al. (1987) Sequences of Proteins of Immunological Interest (National Institutes of Health, Bethesda, Md.; Kabat et al. (1991) Sequences of Proteins of Immunological Interest, Fifth Edition, U.S. Department of Health and Human Services, NIH Publication No. 91-3242; Kontermann and Dubel (eds.) (2001) Antibody Engineering Springer-Verlag, NY 790 pp. (ISBN 3-540-41354-5); Kriegler (1990) Gene Transfer and Expression, A Laboratory Manual, Stockton Press, NY; Lu and Weiner (eds.) (2001) Cloning and Expression Vectors for Gene Function Analysis BioTechniques Press. Westborough, Mass. 298 pp. (ISBN 1-881299-21-X); Langer and Wise (eds.) (1974) Medical Applications of Controlled Release, CRC Pres., Boca Raton, Fla.; Old and Primrose (1985) Principles of Gene Manipulation: An Introduction To Genetic Engineering (3d Ed.) Blackwell Scientific Publications, Boston, Mass. Studies in Microbiology; V.2:409 pp. (ISBN 0-632-01318-4); Sambrook et al. (eds.) (1989) Molecular Cloning: A Laboratory Manual (2d Ed.) Cold Spring Harbor Laboratory Press, NY, Vols. 1-3 (ISBN 0-87969-309-6); Robinson (ed.) (1978) Sustained and Controlled Release Drug Delivery Systems, Marcel Dekker, Inc., NY; Winnacker (1987) from Genes To Clones: Introduction To Gene Technology; VCH Publishers, NY (translated by Horst Ibelgaufts). 634 pp. (ISBN 0-89573-614-4).

Further, the contents of all cited references (including literature references, patents, patent applications, and websites) that maybe cited throughout this application are hereby expressly incorporated by reference in their entirety for any purpose, as are the references cited therein.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A method for treating a subject having psoriatic arthritis (PsA), the method comprising the step of administering to the subject a binding protein that specifically binds IL-17 and TNF-α.

2. The method of claim 1, wherein the binding protein is a dual variable domain immunoglobulin (DVD-Ig) binding protein.

3. The method of claim 1, wherein the subject is resistant to treatment with at least one disease-modifying antirheumatic drug (DMARD).

4. The method of claim 1, wherein the binding protein comprises a heavy chain variable region (VH) for binding TNF-α comprising the amino acid sequence of SEQ ID NO: 5 and a VH for binding IL-17 comprising the amino acid sequence of SEQ ID NO: 7.

5. The method of claim 1, wherein the binding protein comprises the amino acid sequence of SEQ ID NO: 4.

6. The method of claim 1, wherein the binding protein comprises a light chain variable region (VL) for binding TNF-α comprising the amino acid sequence of SEQ ID NO: 10 and a VL for binding IL-17 comprising the amino acid sequence of SEQ ID NO: 12.

7. The method of claim 1, wherein the binding protein comprises the amino acid sequence of SEQ ID NO: 9.

8. The method of claim 1, wherein the binding protein comprises a heavy chain comprising the amino acid sequence of SEQ ID NO: 4 and a light chain comprising the amino acid sequence of SEQ ID NO: 9.

9. The method of claim 8, wherein the binding protein further comprises a heavy chain constant domain and/or a light chain constant domain, wherein the heavy chain constant domain comprises the amino acid sequence of SEQ ID NO: 8; and wherein the light chain constant domain comprises the amino acid sequence of SEQ ID NO: 13.

10. The method of claim 1, the method further comprising the step of administering to the subject a DMARD, wherein the DMARD is selected from the group consisting of methotrexate, sulfasalazine, cyclosporine, leflunomide, hydroxychloroquine, and azathioprine.

11. (canceled)

12. The method of claim 1, wherein the binding protein is administered subcutaneously or intravenously.

13. (canceled)

14. The method of claim 12, wherein the binding protein is administered at a dose selected from the group consisting of about: 0.1 milligram per kilogram of subject weight (mg/kg); 0.3 mg/kg; 1.0 mg/kg; 1.5 mg/kg, 3 mg/kg; and 10 mg/kg.

15. (canceled)

16. The method of claim 12, wherein the binding protein is administered at a dose selected from the group consisting of between 1-25 mg, 25-50 mg, 50-75 mg, 75-100 mg, 100-200 mg, 100-125 mg, 125-150 mg, 150-175 mg, 175-200 mg, 200-225 mg, 225-250 mg, 250-275 mg, 275-300 mg, 300-325 mg, 325-350 mg, and 350-400 mg of the binding protein.

17. The method of claim 12, wherein the binding protein is administered weekly at a dose of about 120 mg or about 240 mg.

18. (canceled)

19. The method of claim 12, wherein the binding protein is administered at least once every: day, every other day, every week, every other week, every month, or every other month.

20. (canceled)

21. The method of claim 10, wherein the subject has been treated with the DMARD for a period of time prior to administration of the binding protein, and the subject is about 1-99% resistant to one or more DMARD activities.

22. The method of claim 10, the method further comprising the step of administering the binding protein after administering the DMARD.

23. The method of claim 1, wherein administering the binding protein improves at least one negative condition in the subject associated with PsA, wherein the at least one negative condition is selected from the group consisting of an autoimmune response, inflammation, stiffness, pain, bone erosion, osteoporosis, joint deformity, joint destruction, a nerve condition, scarring, a cardiac disorder, a blood vessel disorder, high blood pressure, fatigue, anemia, weight loss, abnormal body temperature, a lung disorder, a kidney disorder, a liver disorder, an ocular disorder, a skin disorder, an intestinal disorder, and an infection.

24-27. (canceled)

28. The method of claim 1, wherein administering the binding protein improves a score of at least one PsA metric in the subject wherein the PsA metric is selected from the group consisting of American College of Rheumatology Response Rate (ACR), ACR20, ACR50, ACR70, proportion of subjects achieving Low Disease Activity (LDA), swollen joints, tender joints, patient assessments of pain, global disease activity and physical function, physician global assessment of disease activity and acute phase reactant levels, Disease Activity Score (DAS) 28, Psoriatic Arthritis Disease Activity Score (PASDAS), Psoriasis Area and Severity Index (PASI), assessment of dactylitis, Entheses Sites Comprising the Total Spondyloarthritis Research Consortium of Canada (SPARCC) Enthesitis Index, Self-Assessment of Psoriasis Symptoms (SAPS), quality of life, function and work as measured by the SF36v2, quality of life by self-reporting, change in the quality of life, function and work as measured by Bath AS Disease Activity Index (BASDAI), quality of life, function and work as measured by the Fatigue Numeric Rating Scale, quality of life, function and work as measured by the Sleep Quality Scale, Psoriasis Target Lesion Score, Proportion of subjects achieving ACR70 responder status, and Classification of Psoriatic Arthritis (CASPAR).

29-31. (canceled)

32. The method of claim 1, wherein the method further comprises the step of detecting a modulation in the expression or activity of at least one biomarker, wherein the biomarker is selected from the group consisting of a high-sensitivity C-reactive protein (hsCRP), a matrix metallopeptidase (MMP), a vascular endothelial growth factor (VEGF), a MMP degradation product, C-reactive protein (CRPM), a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), substance P, an inducible Nitric Oxide Synthase (iNOS cartoxin I (CTX-I), cartoxin II (CTX-II), type II collagen neoepitope (TIINE), creatinine, a vimentin, a citrullinated vimentin, an MMP-degraded vimentin, and VICM.

33-36. (canceled)

37. A method for treating a subject having PsA, wherein the subject is resistant to treatment with methotrexate, the method comprising the step of administering to the subject a composition comprising a binding protein that specifically binds both IL-17 and TNF-α, wherein the binding protein is a DVD-Ig protein, and wherein the binding protein comprises at least one heavy chain polypeptide comprising the amino acid sequence of SEQ ID NO: 4 and at least one light chain polypeptide comprising the amino acid sequence of SEQ ID NO: 9, wherein the binding protein is administered weekly or every other week, and wherein the total amount administered to the subject is about 120 mg or 240 mg of the binding protein.

38. A method for treating a subject having PsA, wherein the subject has been or is currently being treated with methotrexate, the method comprising the step of administering to the subject a binding protein that binds TNF-α and IL-17, wherein the binding protein is a DVD-Ig binding protein, wherein the binding protein comprises a heavy chain comprising the amino acid sequence of SEQ ID NO: 4 and a light chain comprising the amino acid sequence of SEQ ID NO: 9, wherein the binding protein is administered at a dose from: 0.005 (milligrams per kilogram) mg/kg to 0.01 mg/kg, 0.01 mg/kg to 0.05 mg/kg, 0.05 mg/kg to 0.1 mg/kg, 0.1 mg/kg to 0.5 mg/kg, 0.5 mg/kg to 1 mg/kg, 1 mg/kg to 1.5 mg/kg, 1.5 mg/kg to 2 mg/kg, 2 mg/kg to 3 mg/kg, 3 mg/kg to 4 mg/kg, 4 mg/kg to 5 mg/kg, 5 mg/kg to 6 mg/kg, 6 mg/kg to 7 mg/kg, 7 mg/kg to 8 mg/kg, 8 mg/kg to 9 mg/kg, or 9 mg/kg to 10 mg/kg of mass of the binding protein to mass of the individual.

39. (canceled)

40. The method of claim 37, wherein the binding protein is administered intravenously.

41. The method of claim 37, wherein the binding protein is administered subcutaneously.

42. The method of claim 37, further comprising the step of administering the binding protein after administering the methotrexate.

43. The method of claim 37, wherein the binding protein is administered at a dosage selected from the group consisting of about 0.1 mg/kg, 0.3 mg/kg, 1.0 mg/kg, 1.5 mg/kg, 3 mg/kg, and 10 mg/kg.

44. The method of claim 37, further comprising the step of identifying an improvement in the subject of severity or duration of at least one symptom associated with the PsA, wherein identifying the improvement comprises using a score, a test, or a metric for PsA.

45. (canceled)

46. The method of claim 44, wherein the score, the test, or the metric is selected from the group consisting of the American College of Rheumatology Response Rate (ACR), ACR20, ACR50, ACR70, the proportion of subjects achieving Low Disease Activity (LDA), swollen joints, tender joints, patient assessments of pain, global disease activity and physical function, physician global assessment of disease activity and acute phase reactant levels, Disease Activity Score (DAS) 28, Psoriatic Arthritis Disease Activity Score (PASDAS), Psoriasis Area and Severity Index (PASI), plaque erythema, plaque scaling, and plaque thickness, assessment of dactylitis, Entheses Sites Comprising the Total Spondyloarthritis Research Consortium of Canada (SPARCC), the Enthesitis Index, Self-Assessment of Psoriasis Symptoms (SAPS), quality of life, function and work as measured by the SF36v2, quality of life by self-reporting, change in the quality of life, function and work as measured by Bath AS Disease Activity Index (BASDAI), quality of life, function and work as measured by the Fatigue Numeric Rating Scale, quality of life, function and work as measured by the Sleep Quality Scale, Psoriasis Target Lesion Score, proportion of subjects achieving ACR70 responder status, and Classification of Psoriatic Arthritis (CASPAR).

47. (canceled)

48. The method of claim 1, wherein the binding protein further comprises at least one constant domain.

49. The method of claim 48, wherein the constant domain is a heavy chain constant domain that comprises the amino acid sequence of SEQ ID NO: 8.

50. The method of claim 48, wherein the constant domain is a light chain constant domain that comprises the amino acid sequence of SEQ ID NO: 13.

51. The method of claim 1, wherein prior to administration the binding protein is lyophilized or crystallized.

52. (canceled)

53. The method of claim 1, wherein the binding protein comprises a conjugate.

54-55. (canceled)

56. The method of claim 37, wherein administering the binding protein reduces a negative condition and/or modulates a biomarker associated with the PsA.

57. The method of claim 1, wherein the binding protein neutralizes TNF-α and IL-17 for a period of time.

58-60. (canceled)

61. A dose of the bispecific binding protein of claim 1 that neutralizes TNF-α and IL-17 sufficient to treat or prevent at least one symptom of PsA.

62-70. (canceled)

71. The method of claim 37, wherein the binding protein further comprises a heavy chain constant domain and a light chain constant domain.

72. The method of claim 71, wherein the heavy chain constant domain comprises the amino acid sequence of SEQ ID NO: 8, and wherein the light chain constant domain comprises the amino acid sequence of SEQ ID NO: 13.

73. The method of claim 38, wherein the binding protein further comprises a heavy chain constant domain and/or a light chain constant domain.

74. The method of claim 73, wherein the heavy chain constant domain comprises the amino acid sequence of SEQ ID NO: 8, and wherein the light chain constant domain comprises the amino acid sequence of SEQ ID NO: 13.

75. The method of claim 37, wherein the binding protein further comprises a variant constant domain.

76. The method of claim 38, wherein the binding protein is administered intravenously.

77. The method of claim 38, wherein the binding protein is administered subcutaneously.


SEQUENCE LISTING (52 sequences)

SEQ ID NO: 1 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 133 aa, Homo sapiens [sequence omitted]

SEQ ID NO: 3 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 257 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSEVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 5 - protein, 121 aa, Artificial Sequence [sequence omitted]

SEQ ID NO: 6 - protein, 10 aa, Artificial Sequence
  Description of Artificial Sequence Synthetic peptide
GGGGSGGGGS

SEQ ID NO: 7 - protein, 126 aa, Artificial Sequence
  Description of Artificial Sequence Synthetic polypeptide
EVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 8 - protein, 330 aa, Artificial Sequence [sequence omitted]

SEQ ID NO: 9 - protein, 225 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGSGGGGSGEIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 10 - protein, 108 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 11 - protein, 9 aa, Artificial Sequence
  Description of Artificial Sequence Synthetic peptide
GGSGGGGSG

SEQ ID NO: 12 - protein, 108 aa, Artificial Sequence
  Description of Artificial Sequence Synthetic polypeptide
EIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 13 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 6 aa, Artificial Sequence
  Description of Artificial Sequence Synthetic peptide
GGGGSG

SEQ ID NO: 15 - protein, 5 aa, Artificial Sequence
  Description of Artificial Sequence Synthetic peptide
GGSGG

SEQ ID NO: 16 - protein, 10 aa, Artificial Sequence
  Description of Artificial Sequence Synthetic peptide
GGGGSGGGGS

SEQ ID NO: 17 - protein, 9 aa, Artificial Sequence
  Description of Artificial Sequence Synthetic peptide
GGSGGGGSG

SEQ ID NO: 18 - protein, 10 aa, Artificial Sequence
  Description of Artificial Sequence Synthetic peptide
GGSGGGGSGS

SEQ ID NO: 19 - protein, 13 aa, Artificial Sequence
  Description of Artificial Sequence Synthetic peptide
GGSGGGGSGGGGS

SEQ ID NO: 20 - protein, 14 aa, Artificial Sequence
  Description of Artificial Sequence Synthetic peptide
GGGGSGGGGSGGGG

SEQ ID NO: 21 - protein, 15 aa, Artificial Sequence
  Description of Artificial Sequence Synthetic peptide
GGGGSGGGGSGGGGS

SEQ ID NO: 22 - protein, 6 aa, Artificial Sequence
  Description of Artificial Sequence Synthetic peptide
ASTKGP

SEQ ID NO: 23 - protein, 13 aa, Artificial Sequence
  Description of Artificial Sequence Synthetic peptide
ASTKGPSVFPLAP

SEQ ID NO: 24 - protein, 5 aa, Artificial Sequence
  Description of Artificial Sequence Synthetic peptide
TVAAP

SEQ ID NO: 25 - protein, 6 aa, Artificial Sequence
  Description of Artificial Sequence Synthetic peptide
RTVAAP

SEQ ID NO: 26 - protein, 12 aa, Artificial Sequence
  Description of Artificial Sequence Synthetic peptide
TVAAPSVFIFPP

SEQ ID NO: 27 - protein, 13 aa, Artificial Sequence
  Description of Artificial Sequence Synthetic peptide
RTVAAPSVFIFPP

SEQ ID NO: 28 - protein, 16 aa, Artificial Sequence
  Description of Artificial Sequence Synthetic peptide
AKTTPKLEEGEFSEAR

SEQ ID NO: 29 - protein, 17 aa, Artificial Sequence
  Description of Artificial Sequence Synthetic peptide
AKTTPKLEEGEFSEARV

SEQ ID NO: 30 - protein, 9 aa, Artificial Sequence
  Description of Artificial Sequence Synthetic peptide
AKTTPKLGG

SEQ ID NO: 31 - protein, 10 aa, Artificial Sequence
  Description of Artificial Sequence Synthetic peptide
SAKTTPKLGG

SEQ ID NO: 32 - protein, 6 aa, Artificial Sequence
  Description of Artificial Sequence Synthetic peptide
SAKTTP

SEQ ID NO: 33 - protein, 6 aa, Artificial Sequence
  Description of Artificial Sequence Synthetic peptide
RADAAP

SEQ ID NO: 34 - protein, 9 aa, Artificial Sequence
  Description of Artificial Sequence Synthetic peptide
RADAAPTVS

SEQ ID NO: 35 - protein, 12 aa, Artificial Sequence
  Description of Artificial Sequence Synthetic peptide
RADAAAAGGPGS

SEQ ID NO: 36 - protein, 27 aa, Artificial Sequence
  Description of Artificial Sequence Synthetic peptide
RADAAAAGGGGSGGGGSGGGGSGGGGS

SEQ ID NO: 37 - protein, 18 aa, Artificial Sequence
  Description of Artificial Sequence Synthetic peptide
SAKTTPKLEEGEFSEARV

SEQ ID NO: 38 - protein, 5 aa, Artificial Sequence
  Description of Artificial Sequence Synthetic peptide
ADAAP

SEQ ID NO: 39 - protein, 12 aa, Artificial Sequence
  Description of Artificial Sequence Synthetic peptide
ADAAPTVSIFPP

SEQ ID NO: 40 - protein, 6 aa, Artificial Sequence
  Description of Artificial Sequence Synthetic peptide
QPKAAP

SEQ ID NO: 41 - protein, 13 aa, Artificial Sequence
  Description of Artificial Sequence Synthetic peptide
QPKAAPSVTLFPP

SEQ ID NO: 42 - protein, 6 aa, Artificial Sequence
  Description of Artificial Sequence Synthetic peptide
AKTTPP

SEQ ID NO: 43 - protein, 13 aa, Artificial Sequence
  Description of Artificial Sequence Synthetic peptide
AKTTPPSVTPLAP

SEQ ID NO: 44 - protein, 6 aa, Artificial Sequence
  Description of Artificial Sequence Synthetic peptide
AKTTAP

SEQ ID NO: 45 - protein, 13 aa, Artificial Sequence
  Description of Artificial Sequence Synthetic peptide
AKTTAPSVYPLAP

SEQ ID NO: 46 - protein, 15 aa, Artificial Sequence
  Description of Artificial Sequence Synthetic peptide
GENKVEYAPALMALS

SEQ ID NO: 47 - protein, 15 aa, Artificial Sequence
  Description of Artificial Sequence Synthetic peptide
GPAKELTPLKEAKVS

SEQ ID NO: 48 - protein, 15 aa, Artificial Sequence
  Description of Artificial Sequence Synthetic peptide
GHEAAAVMQVQYPAS

SEQ ID NO: 49 - protein, 4 aa, Artificial Sequence
  Description of Artificial Sequence Synthetic peptide
FGXG

SEQ ID NO: 50 - protein, 9 aa, Artificial Sequence
  Description of Artificial Sequence Synthetic peptide
CXXXXXXXX

SEQ ID NO: 51 - protein, 5 aa, Artificial Sequence
  Description of Artificial Sequence Synthetic peptide
LEWIG

SEQ ID NO: 52 - protein, 4 aa, Artificial Sequence
  Description of Artificial Sequence Synthetic peptide
WGXG